(19) United States
(12) Patent Application Publication
(10) Pub. No.: US 2008/0103375 A1
(43) Pub. Date: May 1, 2008

(54) Patient monitor user interface

(76) Inventor: Massi E. Kiani, Laguna Niguel, CA (US)

Correspondence Address: LAW OFFICE OF GLENN R. SMITH, 28626 BROOKHILL ROAD, TRABUCO CANYON, CA, 92679-1163, US

(21) Appl. No.: 11/904,046

(22) Filed: Sep. 24, 2007

Related U.S. Application Data
(60) Provisional application No. 60/846,472, filed on Sep. 22, 2006.

(51) Int. Cl. A61B 5/00

(52) U.S. Cl. 600/323; 600/310

(57) ABSTRACT

A patient monitor is configured as a handheld device including a color display, a plurality of user activated soft keys adjacent the color display and a plurality of icons provided on the color display proximate and corresponding to the soft keys. The color display has multiple modes that provide multiple parameter measurements of hemoglobin constituents that may include oxygen saturation displayed in a first color, carboxyhemoglobin displayed in a second color and methemoglobin displayed in a third color, among other parameters.

### CROSS-REFERENCE TO RELATED APPLICATIONS

This application relates to and claims the benefit of prior U.S. Provisional Application No. 60/846,472, filed Sep. 22, 2006, entitled Patient Monitor User Interface, incorporated by reference herein.

### BACKGROUND OF THE INVENTION

Pulse oximetry is a widely accepted continuous and non-invasive method of measuring at least the level of arterial oxygen saturation in blood (SpO₂) and pulse rate. Measurements are taken by placing an optical sensor on a patient, usually on the fingertip for adults and the hand or foot for neonates. The optical sensor used in pulse oximetry has light emitting diodes (LEDs) that transmit optical radiation of red and infrared wavelengths into a tissue site. A detector in the sensor responds to the intensity of the optical radiation after attenuation by pulsatile arterial blood flowing within the tissue site. Based on this response, a processor determines measurements for SpO₂ and pulse rate among other parameters.

A pulse oximeter may also derive and display a plethysmograph, which is a visualization of light absorption at the tissue site versus time. Tissue absorption includes components of static absorption and variable absorption. Static absorption is due to tissue, venous blood and a base volume of arterial blood. Variable absorption is due to the pulse-added volume of arterial blood. Thus, the plethysmograph indicates the arterial blood volume change over time.

Pulse oximeters capable of reading through motion induced noise are disclosed in at least U.S. Pat. Nos. 6,770,028, 6,658,276, 6,650,917, 6,157,850, 6,002,952, 5,769,785, and 5,758,644; low noise pulse oximetry sensors are disclosed in at least U.S. Pat. Nos. 6,985,764, 6,813,511, 6,792,300, 6,256,523, 6,088,607, 5,782,757 and 5,638,818; all of which are assigned to Masimo Corporation, Irvine, Calif. (“Masimo”) and are incorporated by reference herein. Further, physiological monitoring systems that include low noise optical sensors and pulse oximetry monitors, such as any of LNOP® adhesive or reusable sensors, SofTouch™ sensors, Hi-Fi Trauma™ or Blue™ sensors; and any of Radical®, SatShare™, Rad-9™, Rad-5™, Rad-5v™ or PPO+™ Masimo SET® pulse oximeters, are all available from Masimo. Such reading through motion pulse oximeters and low noise sensors have gained rapid acceptance in a wide variety of medical applications, including surgical wards, intensive care and neonatal units, general wards, home care, physical training, and virtually all types of monitoring scenarios.

### SUMMARY OF THE INVENTION

FIG. 1 illustrates a physiological measurement system 100 having a noninvasive sensor 110 attached to a tissue site 1, a patient monitor 120, and an interface cable 130 interconnecting the monitor 120 and the sensor 110. The physiological monitoring system 100 may incorporate pulse oximetry in addition to advanced features, such as a multiple wavelength sensor and advanced processes for determining physiological parameters other than or in addition to those of pulse oximetry, such as carboxyhemoglobin, methemoglobin and total hemoglobin, as a few examples. The patient monitor 120 displays measurements of selected physiological parameters and may also provide visual and audible alarm mechanisms that alert a caregiver when these parameters are outside of predetermined limits. As shown for example, the monitor 120 may display a calculated percentage value for arterial oxygen saturation, e.g. 97%, pulse rate, e.g. 76 beats per minute (bpm), a plethysmograph waveform, a perfusion index, e.g. 3.00, HbCO, e.g. 20%, HbMet, e.g. 1.65% and a perfusion variability index (PVI), e.g. 5, as described in further detail below.

The patient monitor 120 provides a user interface including a display and user interface for controlling display and alarms functions, among other items. Advantageously, the display organizes these many parameters so that a caregiver can readily ascertain patient status using a portable, handheld device. For example, the color display has a color scheme for various parameters that allows ready recognition and distinction of multiple parameters on a portable, handheld device. Also, soft keys and a menu schema allow efficient and intuitive control over display and alarm function on a portable, handheld device.

Patient monitors capable of measuring parameters in addition to SpO₂, such as HbCO, HbMet and total hemoglobin (Hbt) and corresponding multiple wavelength optical sensors are described in at least U.S. patent application Ser. No. 11/367,013, filed Mar. 1, 2006 and entitled Multiple Wavelength Sensor Emitters and U.S. patent application Ser. No. 11/366,208, filed Mar. 1, 2006 and entitled Noninvasive Multi-Parameter Patient Monitor, both assigned to Masimo Laboratories, Irvine, Calif. (Masimo Labs) and both incorporated by reference herein. Further, noninvasive blood parameter monitors and corresponding multiple wavelength optical sensors, such as Rainbow™ adhesive and reusable sensors and RAD-57™ and Radical-7™ monitors for measuring SpO₂, pulse rate, perfusion index, signal quality, HbCO and HbMet among other parameters are also available from Masimo.

FIGS. 2A-C illustrates an instrument that provides the functionality of three patient monitors in one. FIG. 2A illustrates a full-featured handheld patient monitor 201. FIG. 2B illustrates a full-featured standalone patient monitor 205. FIG. 2C illustrates an upgrading patient monitor 209. As shown in FIG. 2A, the handheld 201 contains the majority of the monitor features. Parameter measurement information as well as instrument status data are displayed to a user on a color screen 400. User input is handled through control keys 301 on a front panel. User input and displays are controlled by the handheld 201. A sensor cable 210 inserts into a sensor port 220 on the handheld 201. The handheld 201 is battery powered and can be used either as a transport monitor or as a handheld monitor for spot checks of patient condition. A handheld release button 230 is pressed to pull the handheld 201 out of a docking station 203 (FIG. 2B).

As shown in FIG. 2B, the handheld 201 snaps into a docking station 203 to provide a standalone patient monitor 205. The docking station 203 connects to AC power for standalone operation or handheld battery charging. In one embodiment, a docking station battery is also available. The standalone patient monitor 205 features an analog output, nurse call and a serial output that interfaces to, for example, a printer or computer.

As shown in FIG. 2C, utilizing an interface cable 250, the standalone 205 also interfaces with a sensor port of a multi-parameter patient monitor 270 or other multi-parameter instrument so as to upgrade the instrument from, say, conventional pulse oximetry to motion-tolerate pulse oximetry. The interface cable 250 attaches to the back of the docking station 205.

A handheld pulse oximeter, docking station, standalone pulse oximeter and interface cable are described in U.S. Pat. No. 6,584,336 entitled “Universal/Upgrading Pulse Oximeter,” assigned to Masimo and incorporated by reference herein. Radical® brand pulse oximeters and Radical-7™ brand patient monitors having handheld, docking station and standalone features along with SatShare® and Rainbow™ brand interface cables are available from Masimo.

### BRIEF DESCRIPTION OF THE DRAWINGS

FIG. 1 is a perspective view of a physiological measurement system;

FIGS. 2A-C are perspective views of a three-in-one patient monitor;

FIG. 3 is an illustration of a handheld user interface;

FIGS. 4A-C are illustrations of display views;

FIG. 4A is an illustration of a pleth and signal quality view;

FIG. 4B is an illustration of a numeric view;

FIG. 4C is an illustration of a trend view;

FIGS. 5-8 are illustrations of patient monitor display views, in vertical and horizontal formats;

FIGS. 5A-F are illustrations of five parameter views in a vertical format;

FIGS. 6A-F are illustrations of seven parameter views in a vertical format;

FIGS. 7A-D are illustrations of five parameter views in a horizontal format;

FIGS. 8A-D are illustrations of seven parameter views in a horizontal format;

FIGS. 9A-D are hierarchical charts of soft key icons;

FIG. 10 is a hierarchical chart of a patient monitor user interface;

FIGS. 11A-B are illustrations of horizontal and vertical startup displays;

FIGS. 12A-B are illustrations of horizontal and vertical contrast/brightness adjustment displays;

FIGS. 13A-B are illustrations of horizontal and vertical default displays;

FIGS. 14A-B are illustrations of horizontal and vertical tool bar displays;

FIGS. 15A-B are illustrations of horizontal and vertical menu displays;

FIGS. 16A-B are illustrations of trend displays;

FIGS. 17-29 and 30-41 are horizontal format and vertical format displays,

respectively;

FIGS. 17 and 30 are illustrations of volume adjustment displays;

FIGS. 18A-B and 31A-B are illustrations of alarms displays;

FIGS. 19 and 32 are illustrations of rotate screen displays;

FIGS. 20A-D and 33A-E are illustrations of display-setup displays;

FIGS. 21 and 34A-B are illustrations of general displays;

FIGS. 22 and 35A-B are illustrations of clock setup displays;

FIGS. 23 and 36 are illustrations of “About” displays;

FIGS. 24 and 37 are illustrations of 3D alarm setup displays;

FIGS. 25 and 38A-B are illustrations of configuration displays;

FIGS. 26 and 39 are illustrations of output setup displays;

FIGS. 27 and 40A-B are illustrations of service displays; and

FIGS. 28 and 41 are illustrations of sensitivity displays; and

FIG. 29 is an illustration of trend toggle displays.

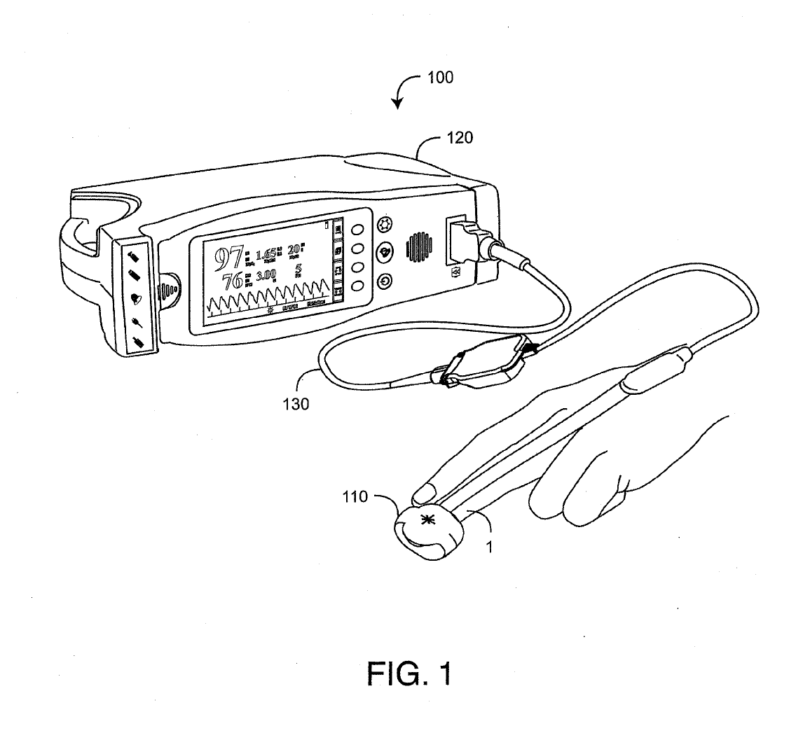

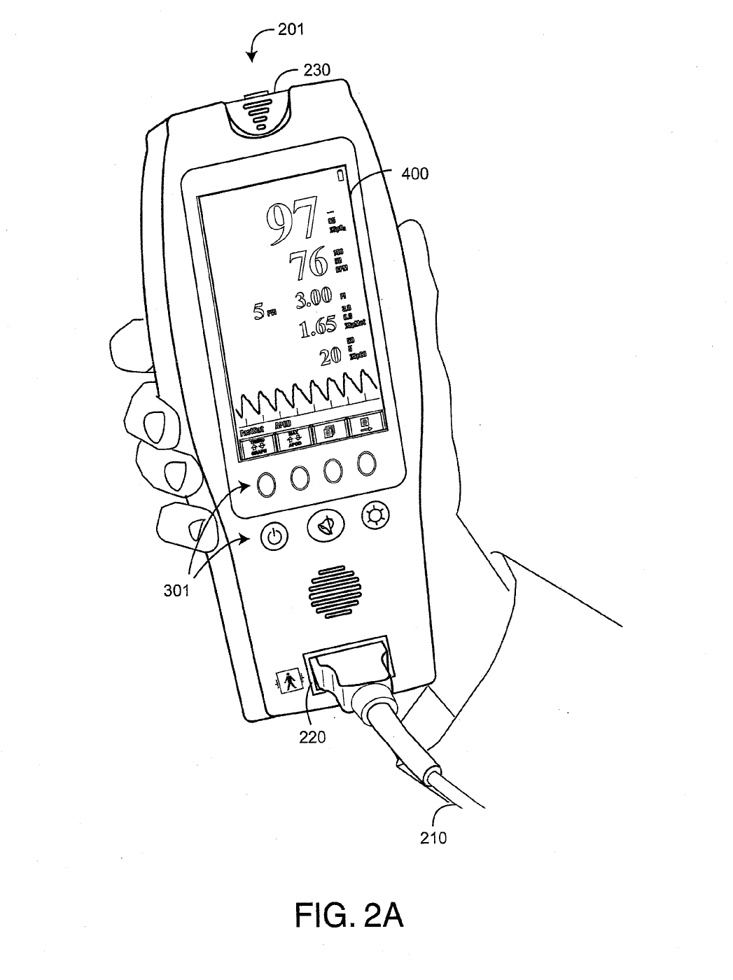

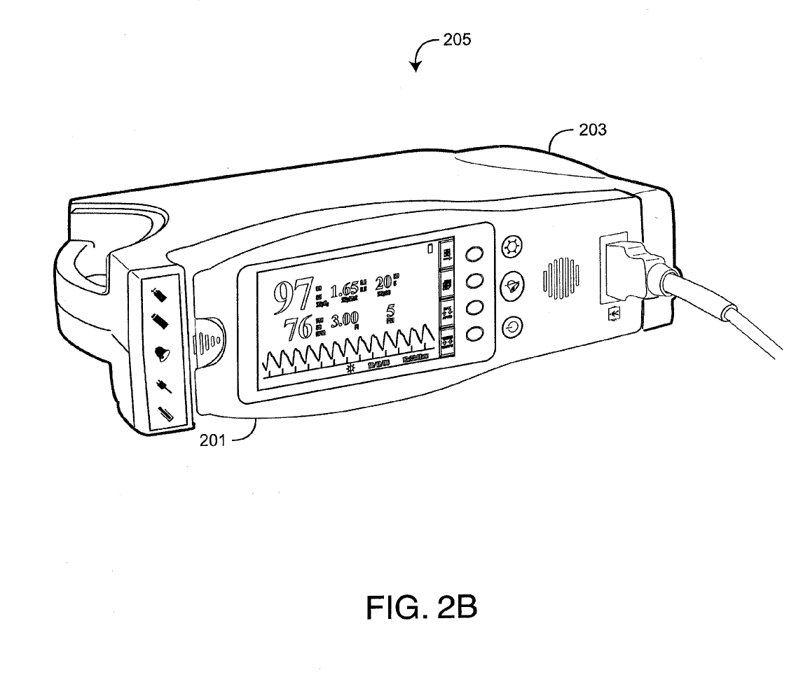

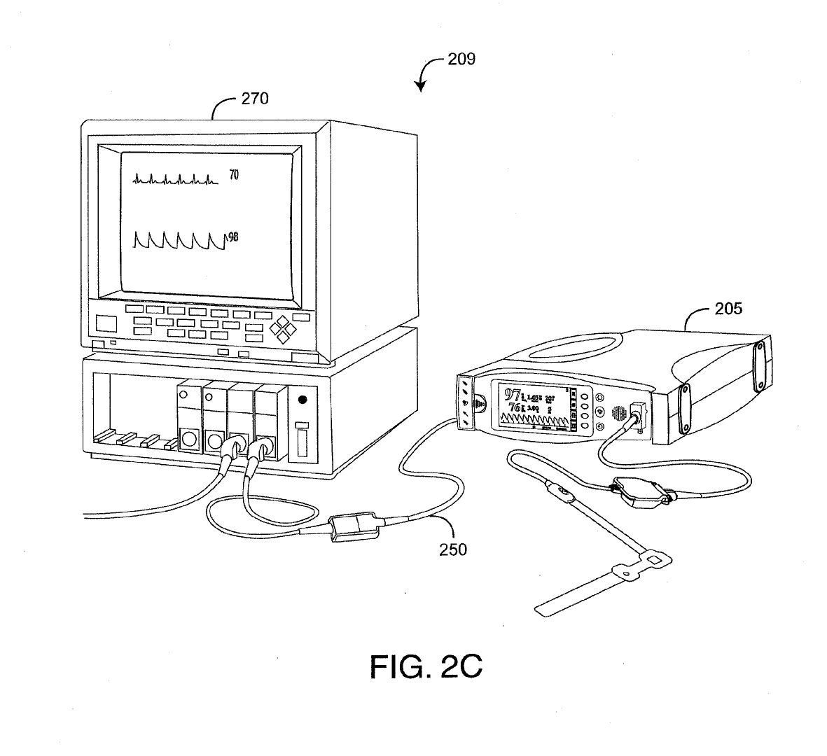

### DETAILED DESCRIPTION OF THE PREFERRED EMBODIMENTS

User Interface

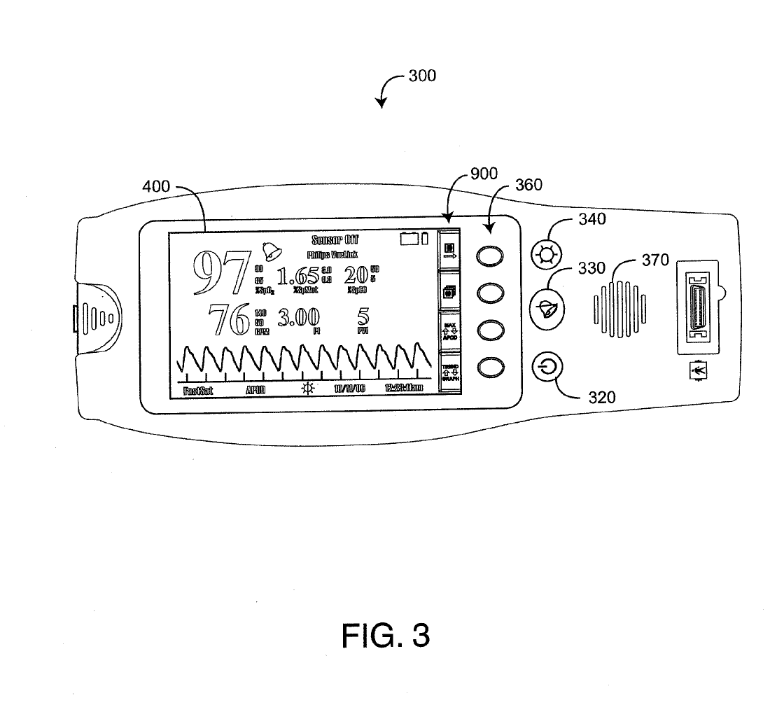

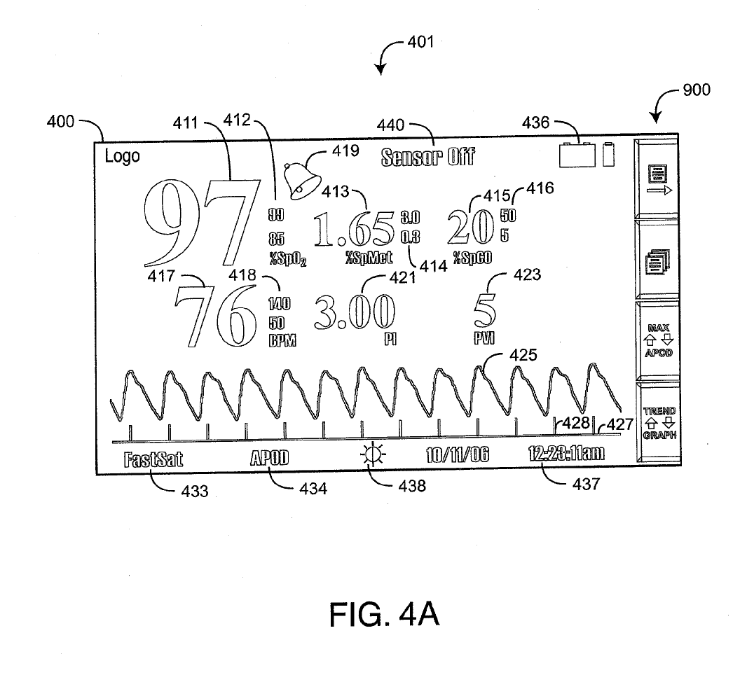

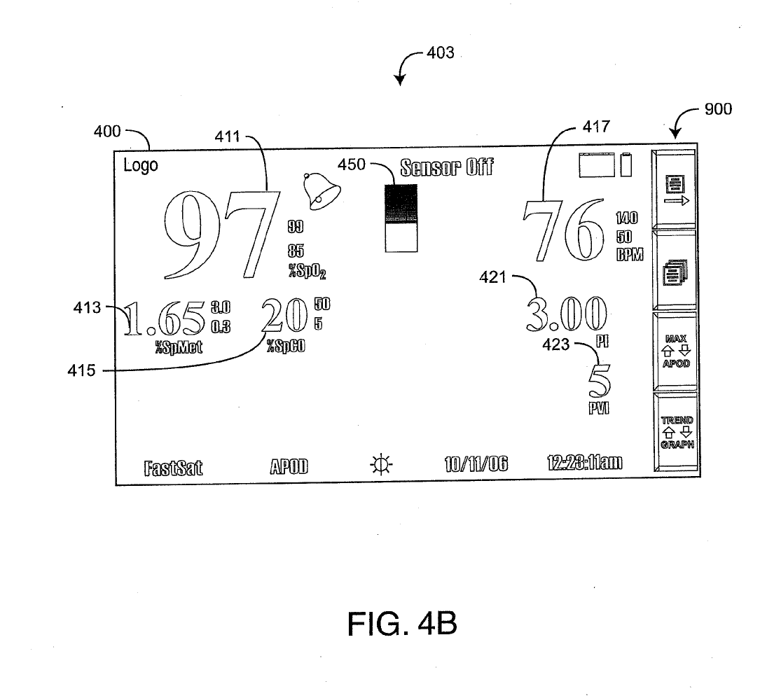

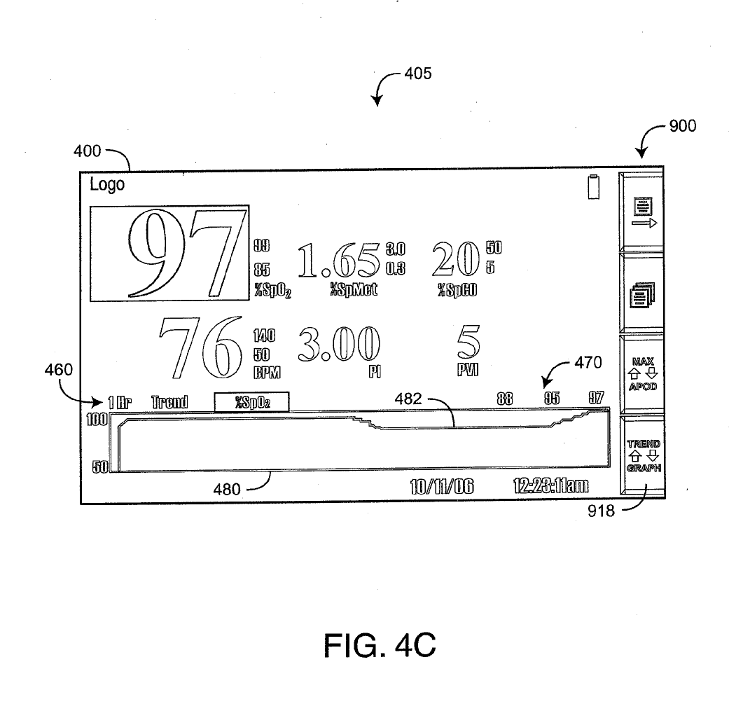

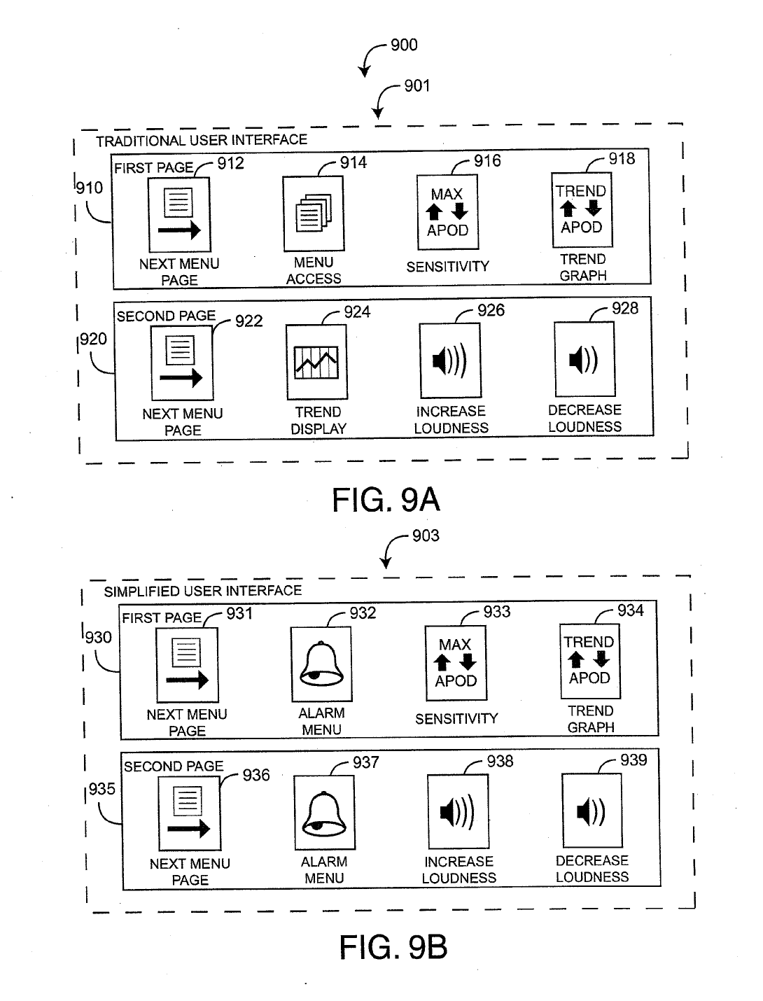

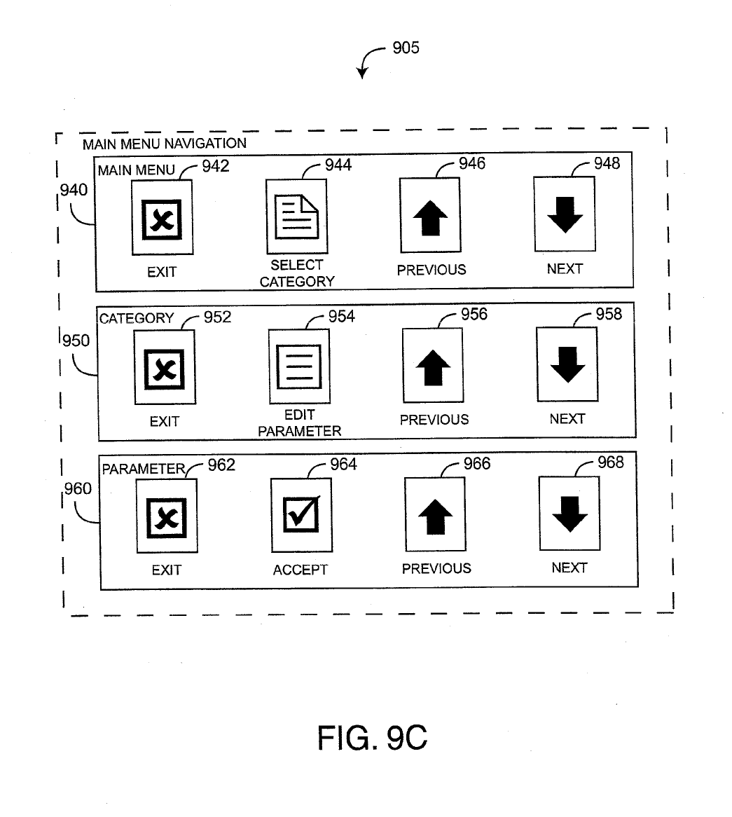

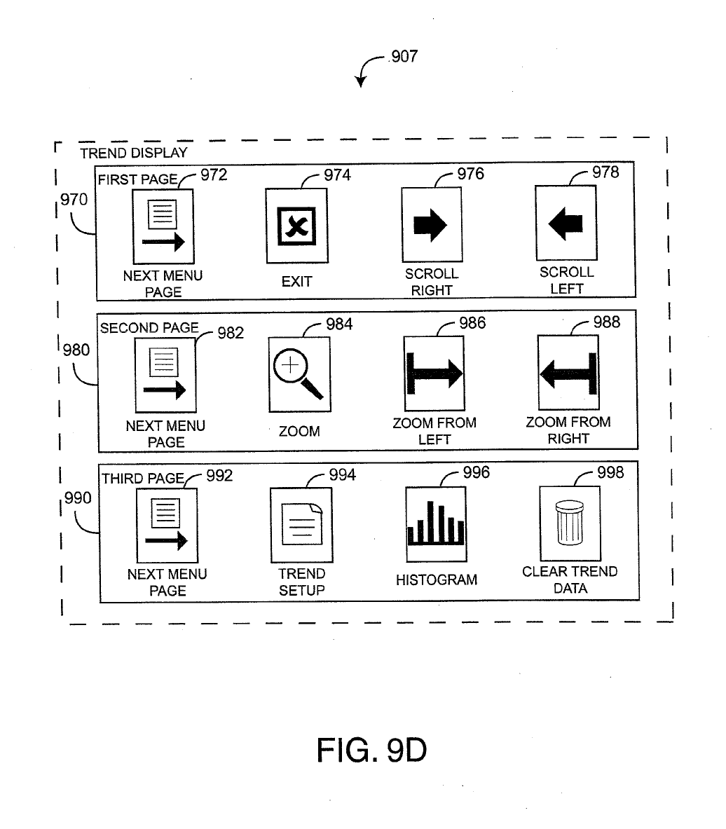

FIG. 3 generally illustrates a handheld user interface 300 having a color display 400, fixed function keys 320-340, programmable function keys (“soft keys”) 360, associated soft key icons 900 and a loudspeaker 370. The color display 400 is described in detail with respect to FIGS. 4A-C, below. The loudspeaker 370 provides an audio indication of alarms. Each of the four soft keys 360, when pressed, select a corresponding one of the soft key icons 900. The soft key icons 900 represent menu items as described with respect to FIGS. 9A-D, below.

The fixed function keys 320-340 include a power/on/off button 320, an alarm silence button 330 and a backlight button 340. The power/on/off button 320 is pressed to turn the instrument on and is held down for more than 2 seconds, then released, to turn the instrument off. The alarm silence button 330 is pressed to temporarily silence patient and low battery alarms. Also, the alarm silence button 330 is pressed when a SENSOR OFF message is flashing, such as when a sensor is removed from a patient, to acknowledge the end of monitoring. In this state, all further alarms are suspended until the monitor starts measuring SpO₂, HbMet, HbCO and pulse rate again. System failure alarms can be silenced by pressing the power/on/off button 320 or the alarm silence button 330. If the power/on/off button 320 does not silence the system fault alarm, the alarm silence button 330 is pressed. The backlight button 340 is pressed to change the illumination level of the backlight. With the AC line power connected, four levels of illumination are available in addition to a no illumination level. In the handheld mode, three levels of illumination are available in addition to a no illumination level. The lowest illumination provides the most efficient battery usage.

Color Display

The color display 400 (FIG. 3) advantageously allows many parameters to be displayed on a relatively small handheld instrument screen. In particular, color selection for parameters, display layout and menu organization, among other features described below, allow users to readily discern parameters of interest. In a medical care environment, for example, the color display enables doctors, nurses, clinicians and other care providers to readily assess patient condition. In an embodiment, key parameters are displayed according to colors represented by the RGB codes listed in Table 1.

TABLE 1
Parameter Colors

| PARAMETER | RGB CODE | COLOR DESCRIPTION |
|---|---|---|
| SpO₂ | 0xFFFFFF | White |
| PR | 0xC0C0C0 | Off White - Silver |
| PVI | 0xC0C0C0 | Off White - Silver |
| Pleth | 0xC0C0C0 | Off White - Silver |
| HbCO | 0xF04000 | Orange-OrangeRed |
| HbMet | 0xF0C000 | Yellow-Gold |
| SpaO₂ | 0x50B050 | Green-LimeGreen |
| Hbt | 0xE01030 | Red-Crimson |

As shown in Table 1, standard available parameters, such as functional oxygen saturation (SpO₂), pulse rate (PR), pulse variability index (PVI) and a plethysmograph waveform (pleth) are displayed in white or an off white color. Other parameters may be optionally purchased as an upgrade (available) or not enabled in a particular instrument and not available (N/A) for display. In an embodiment, two colors are assigned to these optional parameters depending on the available or N/A state of the parameters. Optional parameters and parameter upgrades are described in U.S. patent application Ser. No. 11/757,925 titled Parameter Upgrade System, filed Jun. 4, 2007 and incorporated by reference herein. In a particular embodiment, the display is a Sharp® 4.3″ wide color LCD-TFT device, featuring 24-bit color and 480×272 pixels, such as part number LQ043T3DX02, available from Sharp Microelectronics of the Americas; Camas, Wash.

Display Views

Pleth and Signal IQ View

FIGS. 4A-C illustrates a display 400 that provides different views to a user. FIG. 4A illustrates a pleth and signal quality view 401 having multiple parameter measurements including oxygen saturation (SpO₂) 411, methemoglobin (HbMet) 413, carboxyhemoglobin (HbCO) 415, pulse rate 417, perfusion index (PI) 421 pleth variability index (PVI) 423, a “pleth” waveform 425 and a signal quality waveform 427. Oxygen saturation 411 provides a percentage measure of functional oxygen saturation. Methemoglobin 413 and carboxyhemoglobin 415 provide a percentage measure of these abnormal hemoglobin constituents. Pulse rate 417 displays in beats per minute. When a sensor is not connected to a patient and during pulse search, the display 400 shows dashed lines and a Sensor Off message. Measurements 411, 413, 415, 417 are updated once per second.

The pleth waveform 425 is scaled by a signal strength measure relating the pulsatile signal component (AC) to the non-pulsatile signal component (DC). The signal quality waveform 427 provides a visual indicator of the plethysmogram signal quality. In particular, the signal quality waveform 427 displays the acquired signal quality and the timing of a patient's pulse by a series of vertical lines 428. The height of a particular vertical line of the signal quality waveform 427 indicates the quality of the measured signal. A tall vertical line indicates a high quality signal, while a small vertical line indicates a low quality signal. When the signal quality is very low the accuracy of the measurements may be compromised and the “LOW SIGNAL IQ” system message 440 is displayed, as described above. The signal quality waveform 427 is updated at a frequency of 31.25 times per second. Signal quality may also be shown as a single, pulsating bar 450 (FIG. 4B), as described with respect to the numeric view 403 (FIG. 4B), below. Signal quality is described in U.S. patent application Ser. No. 09/858,114 entitled Pulse Oximetry Data Confidence Indicator, assigned to Masimo and incorporated by reference herein. Perfusion index 421 displays the percentage of pulsatile signal to non-pulsatile signal. The pleth variability index 423 displays the percentage of variation in the pleth waveform as a result of an inhalation and exhalation cycle. A PVI measurement is described in U.S. Prov. Pat. App. No. 60/873,663 entitled Plethysmograph Variability Index, filed Dec. 9, 2006 and incorporated by reference herein.

As shown in FIG. 4A, the pleth and signal quality view 401 also has saturation limits 412, methemoglobin limits 414, carboxyhemoglobin limits 416, pulse rate limits 418 and an alarm status indicator 419. The limits 412, 414, 416, 418 display the upper and lower parameter alarm limits and are displayed next to the associated measurement display 411, 413, 415, 417. When a measured value is outside of an alarm limit 412, 414, 416, the associated measurement display 411, 413, 415 flashes and the alarm will sound. When a pulse rate measured value 417 reaches or exceeds an alarm limit 418, the pulse rate measurement display 417 and the violated limit flash.

The alarm status indicator 419 is a bell symbol that can be shown with or without a slash. It flashes when an alarm condition is present. When the alarm is silenced using the alarm silence button 330 (FIG. 3), an alarm status indicator 419 with a slash and a timer is shown to indicate that the alarm is temporarily silenced. When the alarm is silenced through an “all mute” menu selection, which is permanent until power is cycled or deselected using menu, an alarm status indicator 319 with a slash is shown to indicate that alarm has been silenced.

Also shown in FIG. 4A, the pleth and signal quality view 401 has status messages 433-434 and indicators 436-438. The status messages include a fast signal processing message (“FastSat”) 433 when operating in that mode, and a sensitivity message (“Max” or “APOD”) 434 when operating in normal sensitivity, maximum sensitivity or adaptive probe off detection mode. Fast signal processing is described in U.S. patent application Ser. No. 09/586,845 entitled Variable Mode Averager, assigned to Masimo and incorporated by reference herein.

The indicators include battery status indicators 436, time and date indicators 437, and a brightness level indicator 438. Battery status indicators 436 show the capacity of the handheld and optional docking station batteries. An indicator 436 flashes when less than 15 minutes of battery life is left and the battery needs to be recharged. The docking station battery status indicator is not shown when the optional docking station battery is not present. The time and date indicators 437 display the current time and date. The time is displayed in 12 or 24 hour format. The date is displayed in dd/mm/yyyy or mm/dd/yyyy format. The date and time display format is selected in the clock menu. The brightness level indicator 438 is displayed when utilizing the backlight/contrast button 340 (FIG. 3).

Further shown in FIG. 4A, the pleth and signal quality view 401 has system messages generated by the instrument that are displayed in a system message area 440. Each message and its meaning are described immediately below. An “AMBIENT LIGHT” message indicates that too much light is on the patient (sensor) and inadequate tissue covers sensor detector. A “DEFECTIVE CABLE” message indicates that the oximeter cannot identify the connected cable or the cable has failed. An “INCOMPATIBLE SENSOR” message indicates an improper sensor. An “INVALID SENSOR” message indicates the oximeter cannot identify the connected sensor, which can be due to a broken sensor cable wire, inoperative LEDs or a faulty detector. A “LOW BATTERY” message indicates that the battery charge is low, signaling that the handheld be placed into the docking station to be powered with AC line power or that the battery be replaced. A “LOW PERFUSION” message indicates that the signal is too small. A “LOW SIGNAL IQ” message indicates a low signal quality. A “LOW SpCO CONF” message indicates a HbCO measurement reading is obscured. A “LOW SpMet CONF” message indicates a HbMet measurement reading is obscured. A “SPEAKER FAILURE” message indicates unit requires service. A “NO CABLE” message indicates cable is not attached or not fully inserted into the connector. A “NO SENSOR” message indicates that a sensor is not fully inserted into the connector, which may be due to an incorrect sensor, or a defective sensor or cable, or that the sensor is inserted upside down. A “PULSE SEARCH” message indicates that the instrument is searching for patient's pulse. A “SENSOR CALIBRATING” message indicates the instrument is checking the sensor for proper functioning and performance. A “SENSOR OFF” message indicates that a sensor is off the patient and should be reattached. A “SERVICE REQUIRED” message indicates an internal failure and that the instrument requires service. An “UNRECOGNIZED CABLE” message indicates an improper cable and the cable should be replaced.

Numeric View

FIG. 4B illustrates a numeric view 403 having the features of the pleth view 401 (FIG. 4A) without the pulse waveform 425 (FIG. 4A). In particular, the numeric view prominently displays multiple parameter measurements such as oxygen saturation 411, methemoglobin 413, carboxyhemoglobin 415, pulse rate 417, perfusion index 421 and pleth variability index 423. Further, the numeric view 403 features a signal quality bar 450 having a pulsating height that is responsive to the patient's arterial pulse and to signal quality. Signal quality is described with respect to FIG. 4A, above. Specifically, the bar height pulses coincide with peaks of an arterial pulsation and the bar height indicates signal quality, with a generally small bar height corresponding to low signal quality and a generally large bar height corresponding to high signal quality.

The PI 421 display provides a relative numeric indication of the pulse strength at the monitoring site. PI 421 is a calculated percentage between the pulsatile signal and non-pulsatile signal of arterial blood moving through the site. PI 421 may be used to find the best perfused site and to monitor physiological changes in the patient and displays an operating range of 0.02 percent to 20.00 percent.

Trend View

FIG. 4C illustrates a trend view 405 that allows a user to quickly check the trend of each parameter by allowing the user to step through and select the desired parameter. The trend view 405 has a first top line of information 460, a second top line of information 470, a trend graph 480 and soft key icons 900 including a trend graph icon 918. The first top line of information 460 shows the time scale of the trend graph followed by the selected parameter. The parameter's numeric value is highlighted. The second top line of information 470 shows the minimum, average, and maximum measurement of the selected parameter contained in the displayed data set, excluding zero measurements. The trend graph 480 shows the desired parameter measurements displayed versus time and accuracy range. Lines on the trend graph 482 indicate minimum and maximum values of the parameter. The trend graph icon 918 initiates the trend graph display. Repeatedly selecting the trend graph icon 918 by repeatedly pressing down on the associated soft key button 360 (FIG. 3) steps through each parameter. Once the parameter is selected, the numeric value is highlighted and the selected parameter is displayed above the trend graph 480.

In the display views 401-405, the soft key icons 900 can be traditional user interface soft key icons 901 (FIG. 9A), simplified user interface soft key icons 903 (FIG. 9B), main menu soft key icons 905 (FIG. 9C) or trend display icons 907 (FIG. 9D), as described below.

Multiple Parameter Display Views

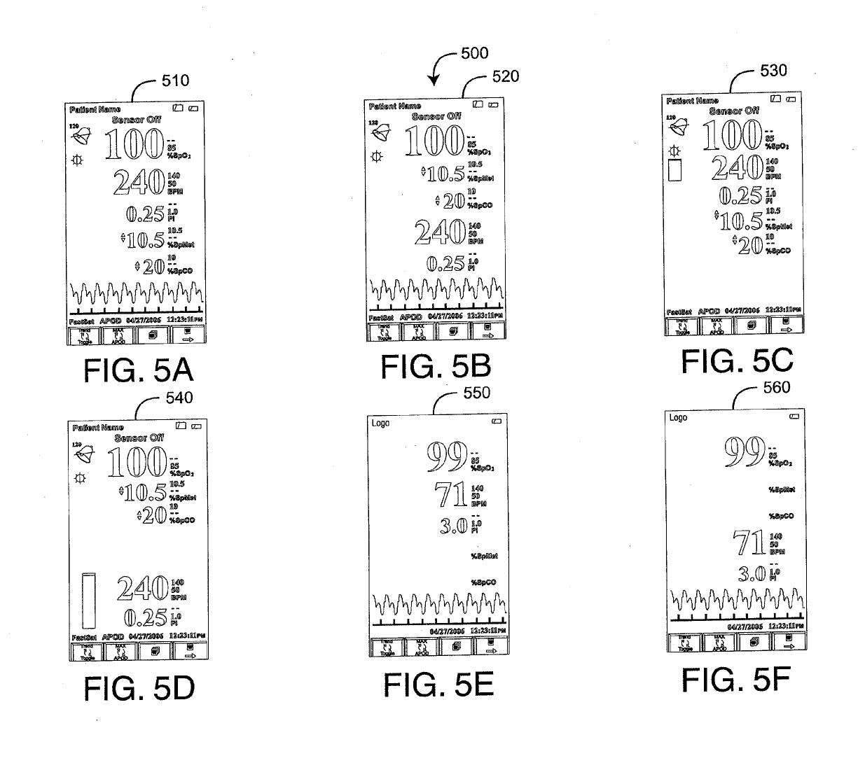

FIGS. 5-8 illustrate multiple parameter display views in vertical and horizontal formats. FIGS. 5A-F illustrate five parameter vertical views 500 including an a pleth and signal IQ view 510, an alternative pleth and signal IQ view 520, a numeric view 530, an alternative numeric view 540, a non-multiple parameter view 550 and an alternative non-multiple parameter view 560. The pleth and signal IQ view 510, 520 shows five parameters and a pulse waveform. The numeric view 530, 540 shows five parameters only. The non-multiple parameter views 550, 560 show parameter measurements of SpO₂, BPM and PI only, along with a pulse waveform.

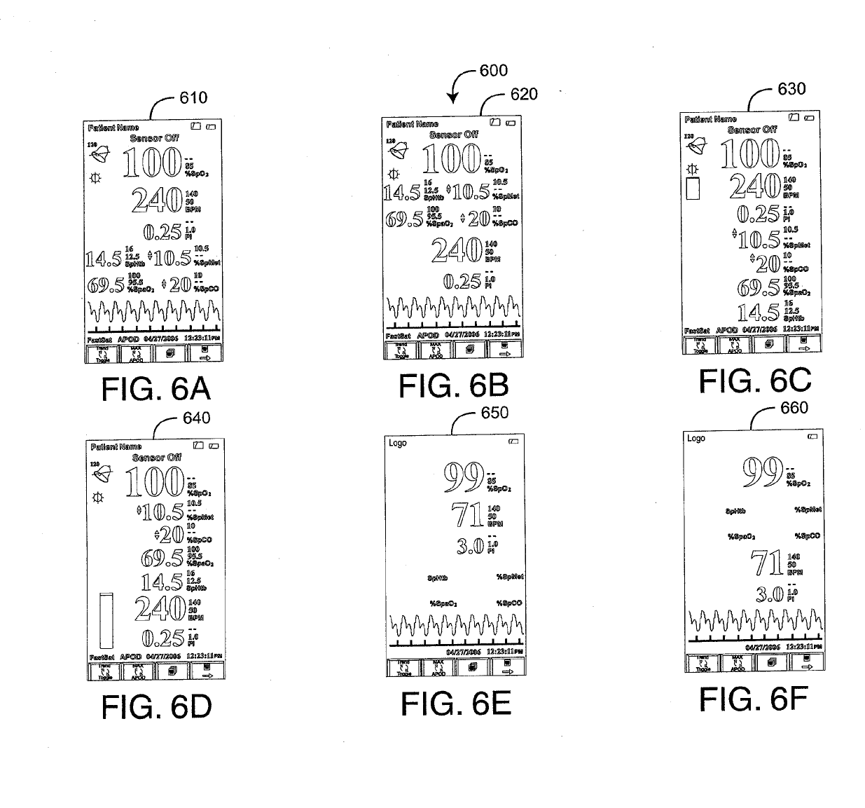

FIGS. 6A-F illustrate seven parameter vertical views 600 including a pleth and signal IQ view 610, an alternative pleth and signal IQ view 620, a numeric view 630, an alternative numeric view 640, a non-multiple parameter view 650 and an alternative non-multiple parameter view 660.

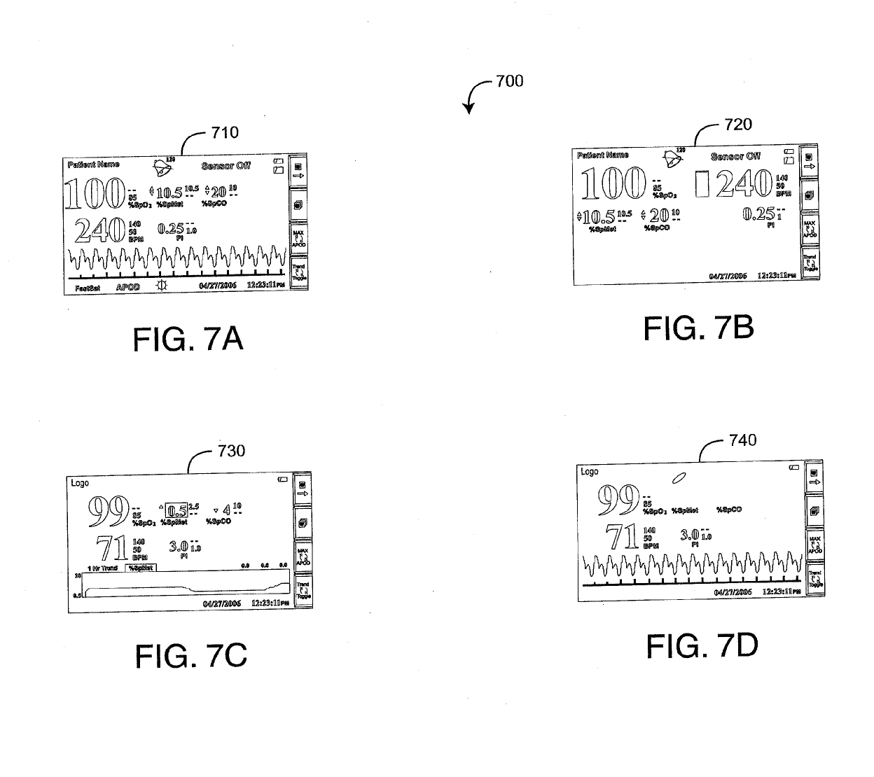

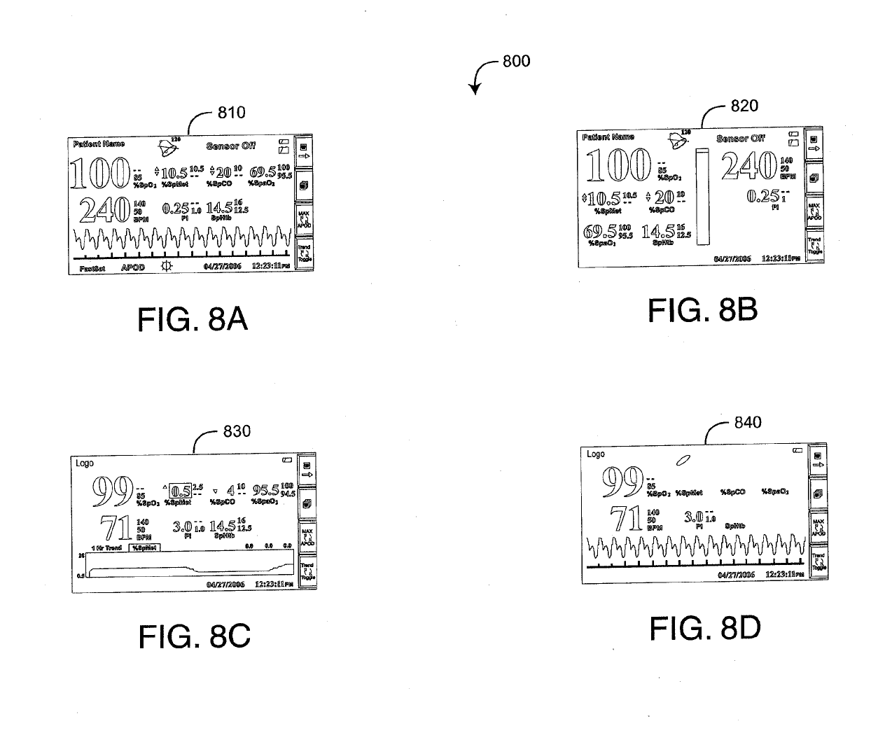

FIGS. 7A-D illustrate five parameter horizontal views 700 including a pleth and signal IQ view 710, a numeric view 720, a trend view 730 and a non-multiple parameter view 740. The pleth and signal view 710 shows five parameters and a pulse waveform. The numeric view 720 shows five parameters only. The trend view 730 shows five parameters and a trend graph of a selected parameter. The non-multiple parameter view 740 shows parameter measurements of SpO2, BPM and PI only, along with a pulse waveform. FIGS. 8A-D illustrate seven parameter horizontal views 800 including a pleth and signal IQ view 810, a numeric view 820, a trend view 830 and a non-multiple parameter view 840.

Soft Keys

FIGS. 9A-D are hierarchical charts of soft key icons 900 corresponding to the soft key buttons 360 (FIG. 3). A soft key icon is selected by pressing and releasing the soft key button to the right of the icon in a horizontal display or underneath the icon in a vertical display. Four icons are shown on the right side or bottom of the display. FIG. 9A illustrates a set of traditional user interface soft key icons 901 including first page icons 910 and second page icons 920. As shown in FIG. 9A, the first page icons 910 include next menu page 912, menu access 914, sensitivity 916 and trend graph 918. Next menu page 912 is selected to access the second page of selections 920. Menu access 914 is selected to enter the main menu. Sensitivity 916 is selected to toggle between the normal, APOD and maximum sensitivity modes. The normal sensitivity setting is used for typical monitoring purposes. The APOD setting is used where there is a high probability of the sensor becoming detached. The maximum sensitivity setting is used for patients with low perfusion or when the low perfusion message is displayed on the screen in APOD or normal sensitivity mode. The default is APOD. Trend graph 918 is selected to alternate between SpO₂, BPM, PI, HbMet, HbCO and PVI Quick Trend displays. Also shown in FIG. 9A, the second page icons 920 include next menu page 922, trend display 924, increase loudness 926 and decrease loudness 928. Next menu page 922 is selected to access the first page of selections 910. Trend display 924 is selected to show the trend data on the display. Increase loudness 926 is selected to increase the volume of the pulse beep. Decrease loudness 928 is selected to decrease the volume of the pulse beep.

FIG. 9B illustrates a set of simplified user interface soft key icons 903 including first page icons 930 and second page icons 935. By enabling the simplified user interface, users are exposed to only the most common monitor features, while the all the remaining settings remain available behind password protection. As shown in FIG. 9B, the first page icons 930 include next menu page 931, alarm menu 932, sensitivity 933 and trend graph 934. Next menu page 931 is selected to access the second page of selections 935. Alarm menu 932 is selected to enter the alarm settings menu. Sensitivity 933 and trend graph 934 are selected to function as described in the traditional user interface first page icons 910. Also shown in FIG. 9B, the second page icons 935 include next menu page 936, alarm menu 937, increase loudness 938 and decrease loudness 939. Next menu page 936 is selected to access the first page of selections 930. Alarm menu 937, increase loudness 938 and decrease loudness 939 are selected to function as described, above.

FIG. 9C illustrates a set of main menu selection icons 905 including main menu icons 940, category icons 950 and parameter icons 960. When the main menu is accessed, the plethysmograph and signal IQ waveform displays are replaced with the main menu items. The touch key icons, displayed along the right edge of the LCD display, are also replaced by the menu access icons. When the main menu is accessed the monitor remains functional and the saturation and pulse rate numbers will continue to be displayed.

As shown in FIG. 9C, main menu icons 940 include exit 942, select category 944, previous 946 and next 948. Exit 942 is selected to exit the main menu. Select category 944 is selected to select the highlighted menu item and enter the next level menu. Previous 946 is selected to scroll through the menu items without selecting them. Next 948 is also selected to scroll through the menu items without selecting them. Once a menu item is highlighted, the menu is entered by selecting the select category icon 944.

Also shown in FIG. 9C, once a menu category has been selected, category icons 950 including exit 952, edit parameter 954, previous 956 and next 958 are displayed. Exit 952 is selected to exit the menu category and return to the previous menu. Edit parameter 954 is selected to select the highlighted parameter for editing. Previous 956 and next 958 are as described above. Once a parameter has been selected for editing, parameter icons 960 including exit 962, accept 964, previous 966 and next 968 are displayed. Exit 962 is selected to exit the parameter without making the new selections permanent. Accept 964 is selected to save the changes. Previous 956 is selected to increase or toggle the parameter settings. Next 968 is selected to decrease or toggle the parameter settings.

FIG. 9D illustrates a set of trend display icons 907 having three pages of menu selections including first page icons 970, second page icons 980 and third page icons 990. These menu screens are not accessible when using the simplified user interface. The first page icons 970 include next menu page 972, exit 974, scroll right 976 and scroll left 978. Next menu page 972 is selected to access the next page of menu selections. Exit 974 is selected to return to the normal display screen. Scroll right 976 is selected to scroll through the data set. The display scrolls by ½ the selected time scale. For example, if a 2 hr display view is selected, then selecting the scroll right icon will scroll the displayed data by 1 hr to the right. Scroll left 978 is selected to scroll through the data set. The display scrolls by ½ the selected time scale. For example, if a 2 hr display view is selected, then selecting the scroll left icon will scroll the displayed data by 1 hr to the left. Also shown in FIG. 9D, the second page icons 980 include next menu page 982, zoom 984, zoom from left 986 and zoom from right 988. Next menu page 982 is selected to access the next page of menu selections. Zoom 984 is selected to change the time scale of the trend view. The available time scales are 24 hrs, 12 hrs, 8 hrs, 4 hrs, 2 hrs, 1 hr, 30 minutes, 10 minutes, 1 minute and 20 seconds. The Zoom icon uses the last recorded data point as the zoom reference point. In other words, the last recorded data point is always shown as the right-most data point on the display. Zoom from left 986 is selected to change the time scale from the left. Zoom from right 988 is selected to change the time scale from the right.

Further shown in FIG. 9D, the third page icons 990 include next menu page 992, trend setup 994, histogram 996 and clear trend data 998. Next menu page 992 is selected to return to the first page of menu selections. Trend setup 994 is selected to enter a trend setup menu. Histogram 996 is selected to display the selected data set that is shown in the trend view in histogram format. Clear trend data 998 is selected to clear the data stored in the trend memory.

User-Based Displays

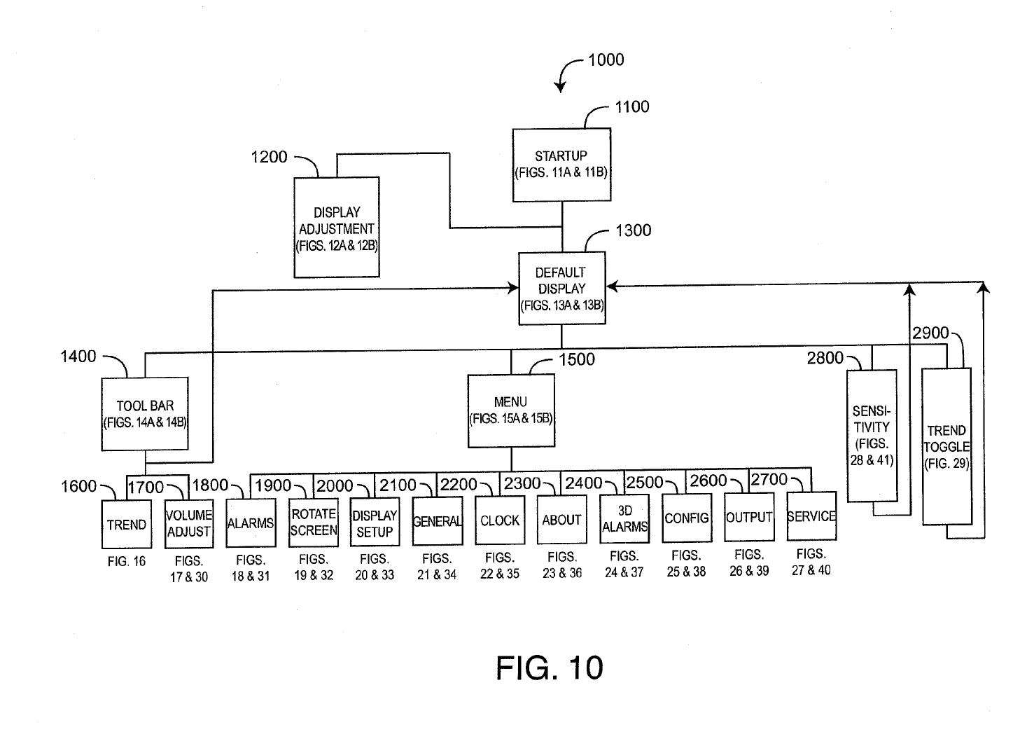

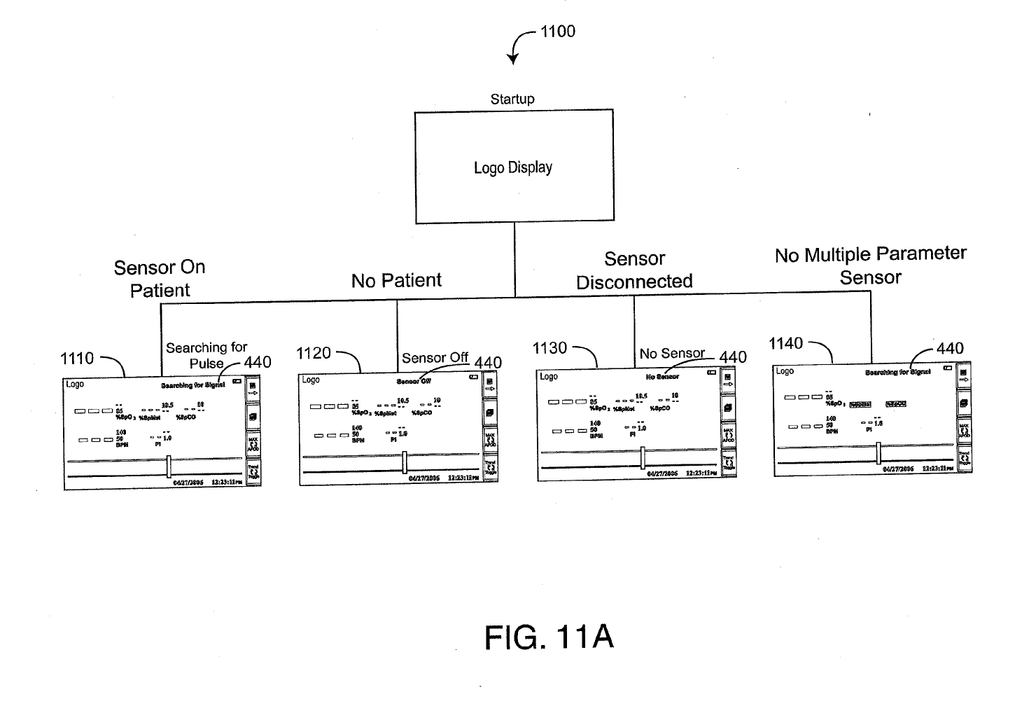

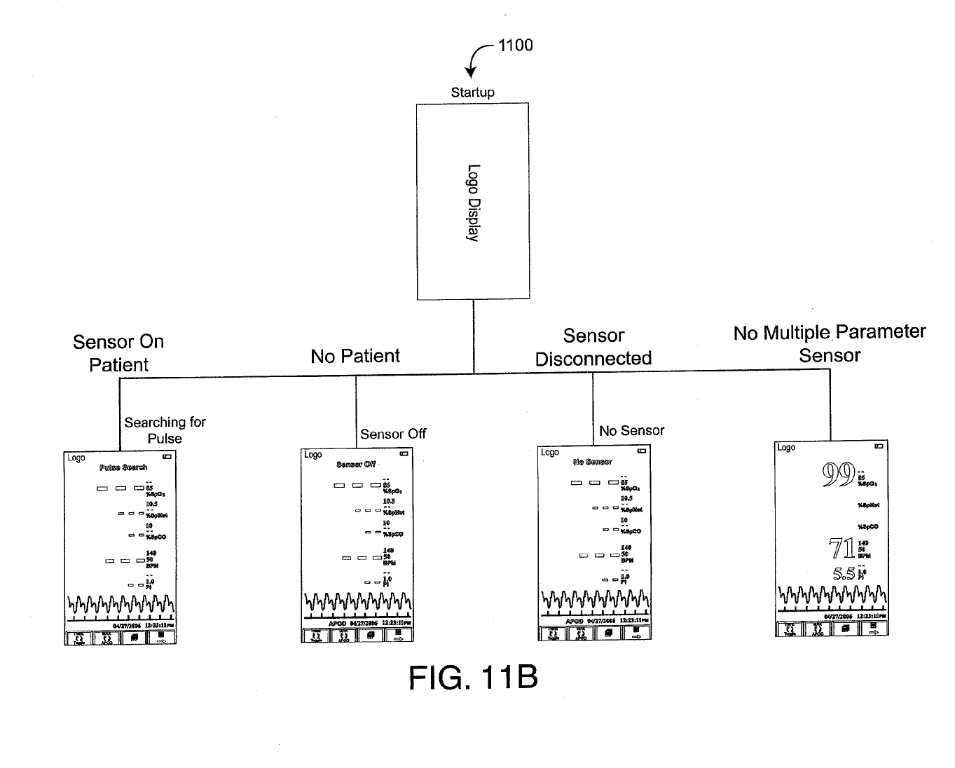

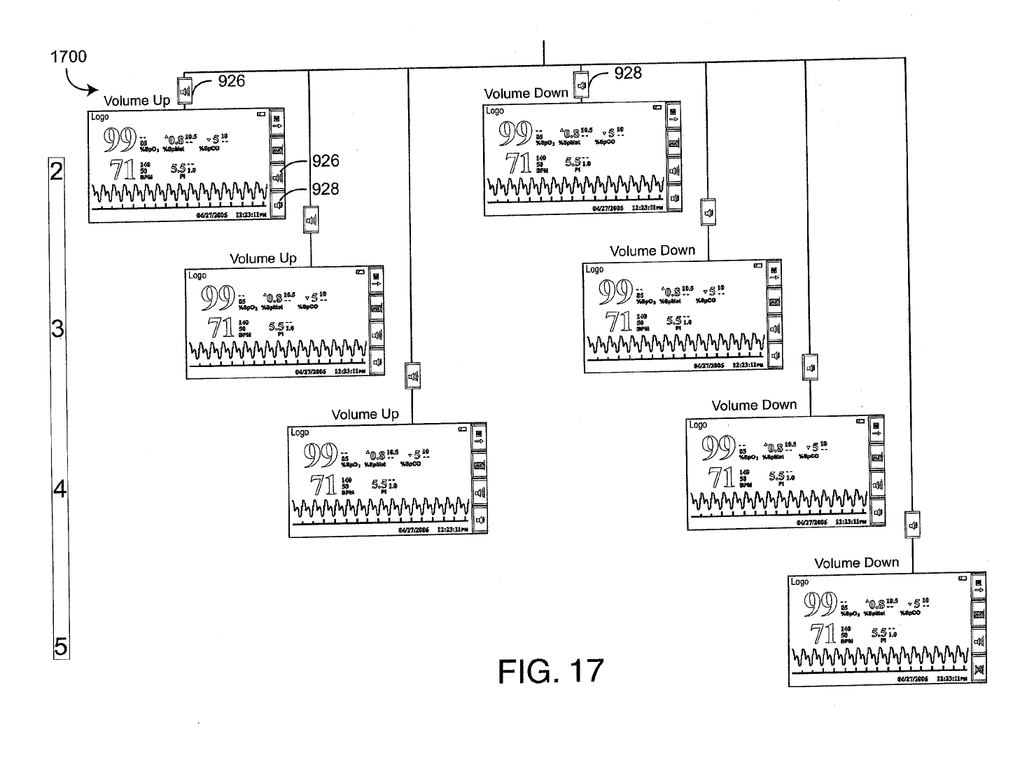

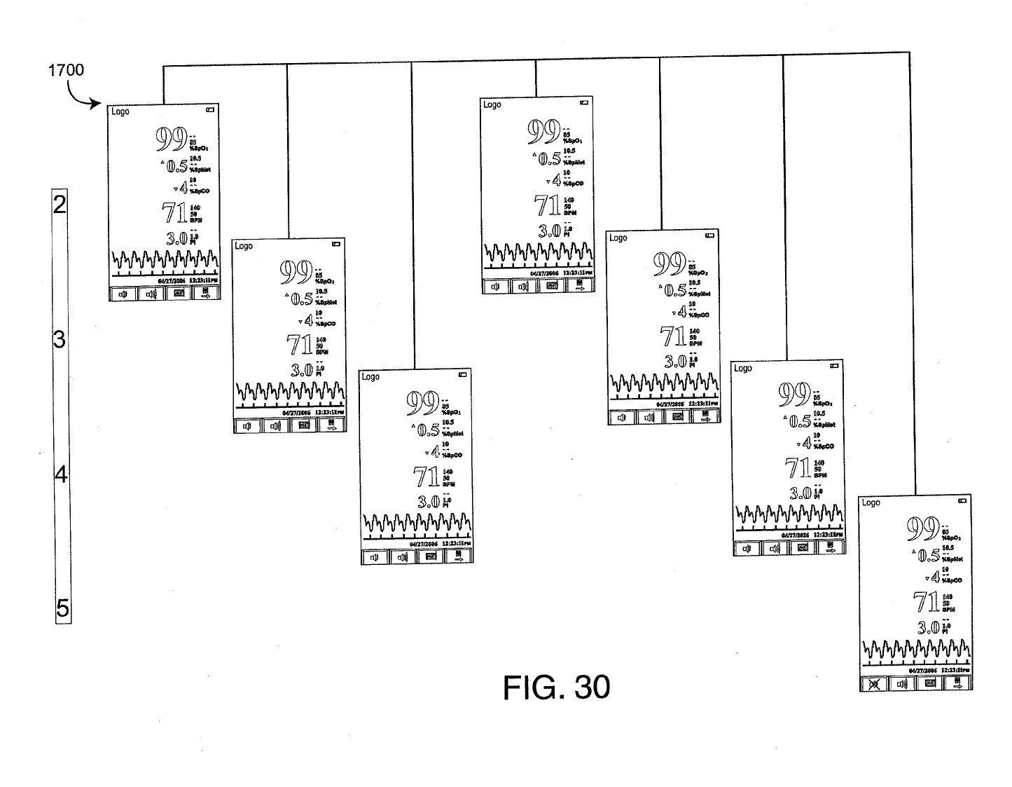

FIG. 10 provides a hierarchical overview 1000 of a plurality of user-based displays (FIGS. 11-41). The hierarchy levels, proceeding from top-to-bottom, range from general function selections to detailed displays according to the number of keystrokes from a startup display to a currently presented display. Specifically, the display transitions illustrated by vertical connecting lines between displays in the following figures are assumed to flow downward on the page according to the number of keystrokes. That is, down arrows are not shown in the figures, but up arrows illustrate how a user returns to more general displays from more specific displays. The keystroke numbers from the startup display are listed near the left margin of each page. Also illustrated with the vertical connecting lines are the associated soft key icons or fixed-function keys that the user presses to make the indicated transition to the next illustrated display. FIGS. 17-28 illustrate horizontal format displays. FIGS. 30-41 illustrate equivalent vertical format displays.

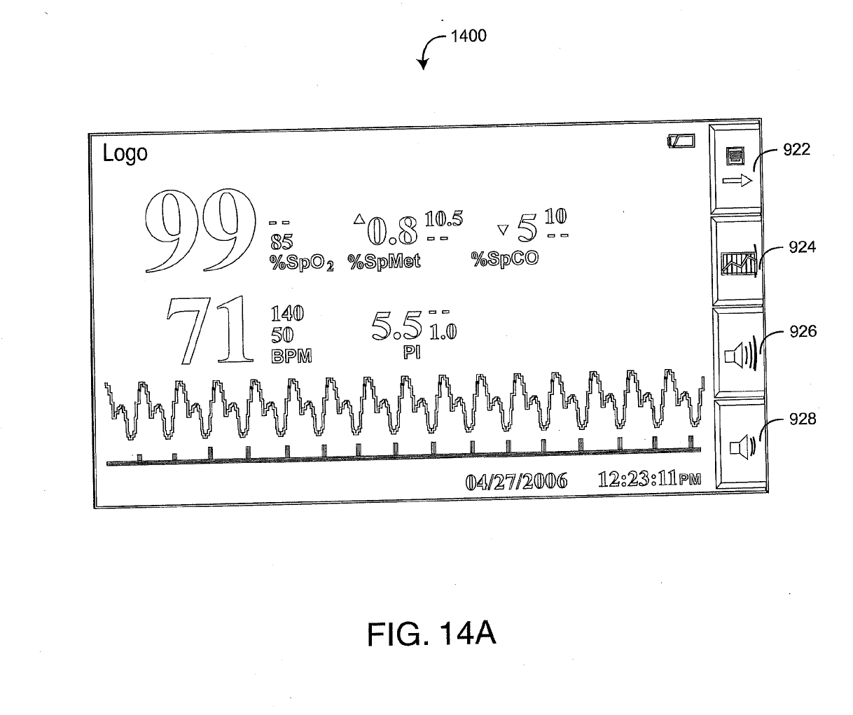

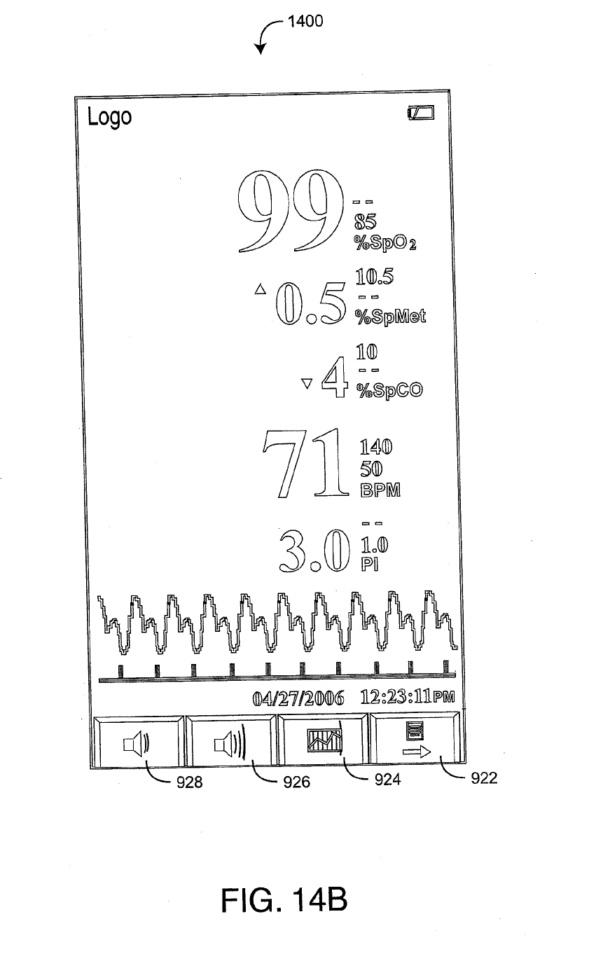

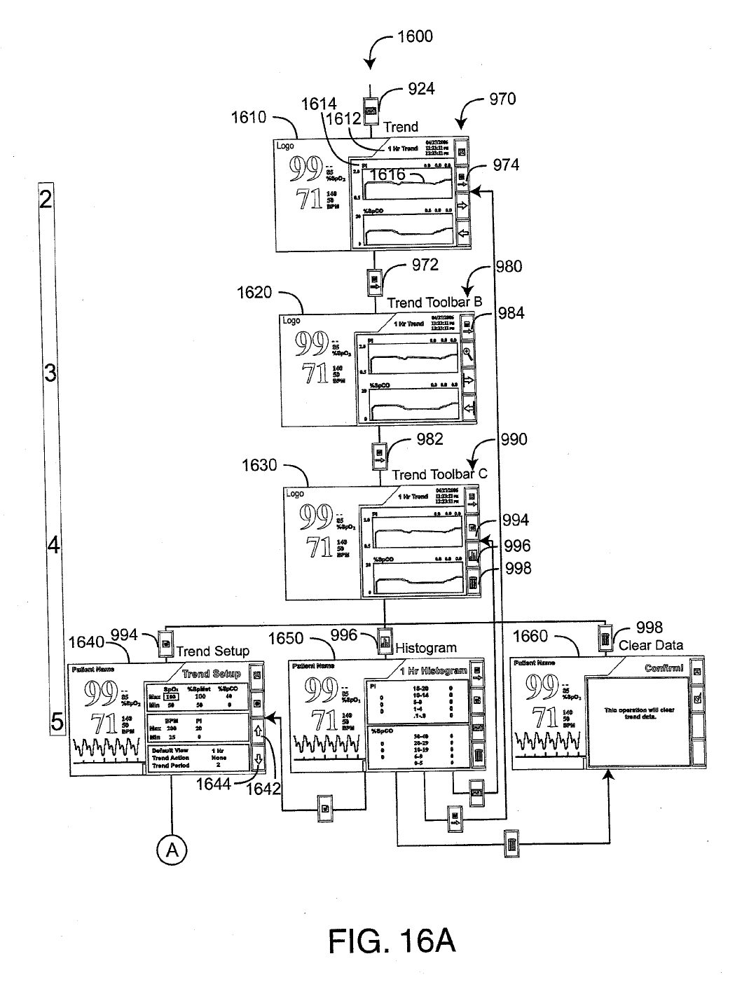

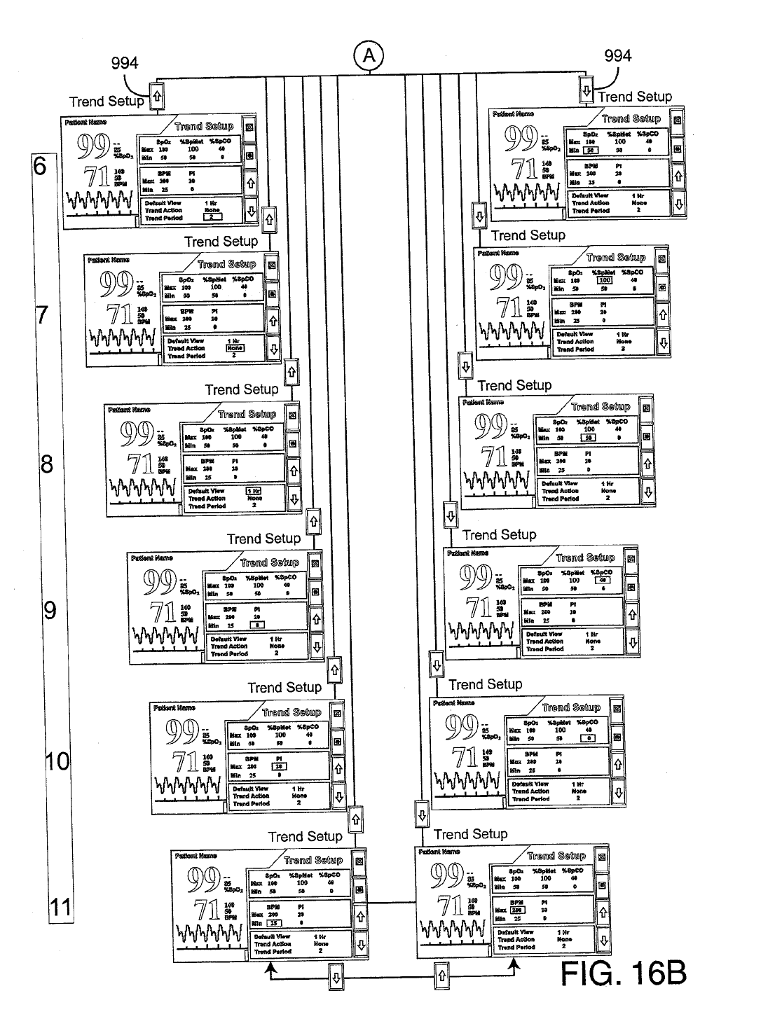

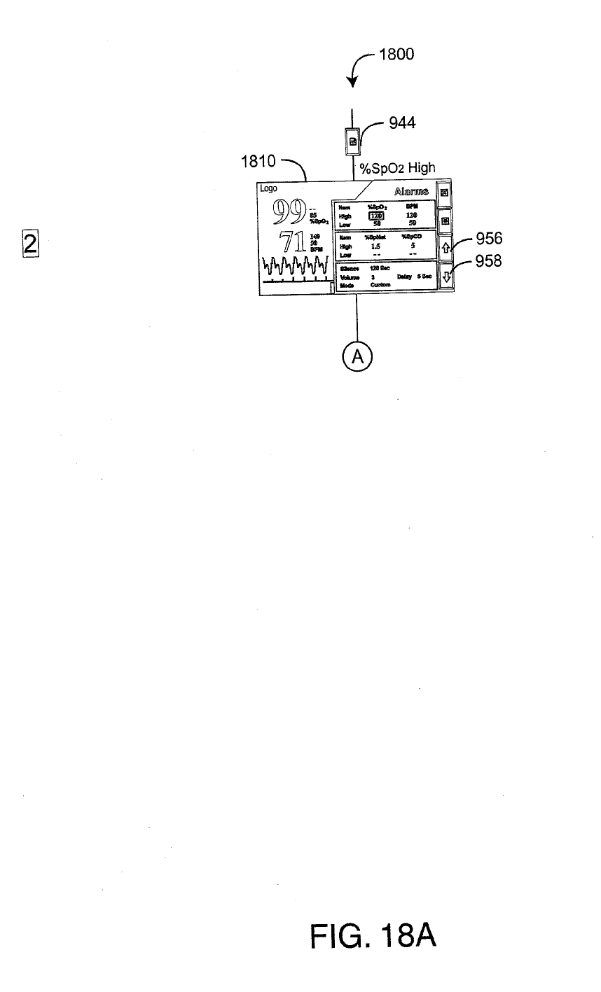

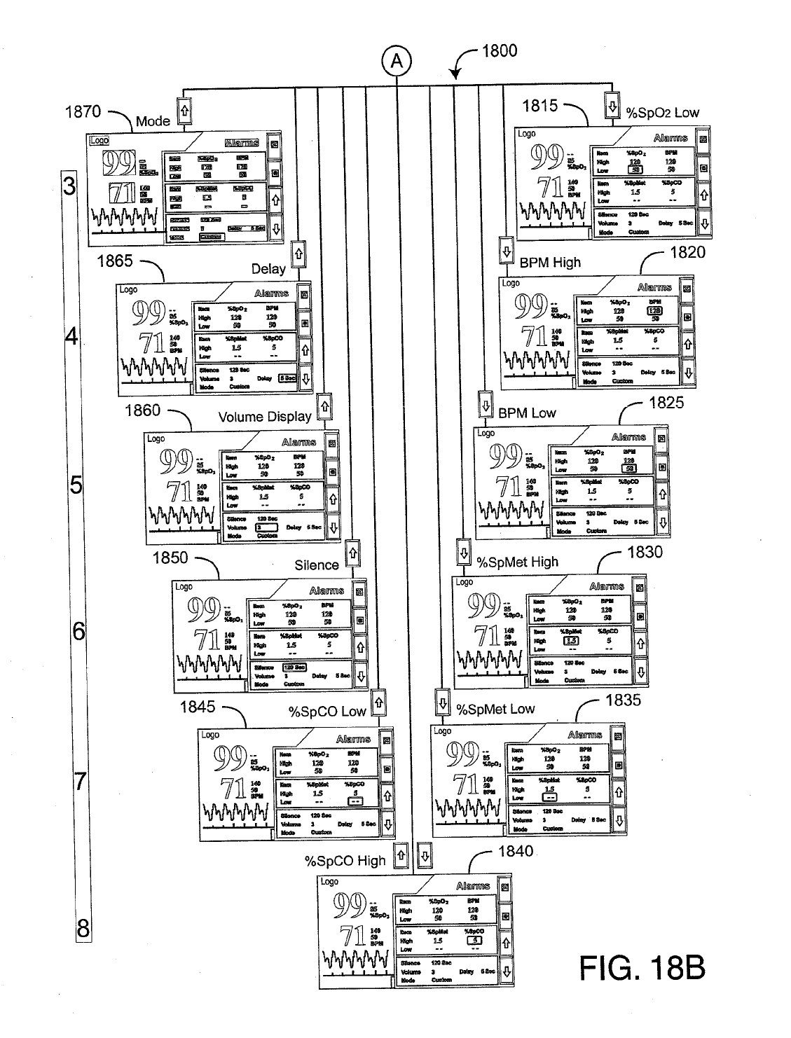

As shown in FIG. 10, from a startup display 1100 a user can transition to displays for contrast and brightness adjustment 1200 or to a default display 1300, as described in detail with respect to FIGS. 11-13, below. The default display 1300 provides for selections of a tool bar display 1400, a menu display 1500, a detection sensitivity display 2800 and a trend toggle display 2900, as described in detail with respect to FIGS. 14-15, 28-29 and 41. The tool bar display 1400 allows selection of a trend display 1600 and a volume adjustment display 1700, as described in detail with respect to FIGS. 16-17 and 30. The menu display 1500 lists selections of alarms 1800, rotate screen 1900, display setup 2000, general 2100, clock 2200, about 2300, 3D alarms 2400, configuration 2500, output 2600 and service displays 2700, as described with respect to FIGS. 18-27 and 31-40.

FIGS. 11A-B illustrate the startup displays 1100 in horizontal and vertical formats. The startup displays 1100 have a sensor on view 1110, a sensor off view 1120, a no sensor view 1130 and a non multiple parameter sensor view 1140. Each of the views 1110, 1120, 1130, 1140 has initiated parameters without measurements and a system message area 440 indicating a sensor position. The sensor on view 1110 is shown when a sensor is on a patient and the system message area 440 indicates searching for signal. The sensor off view 1120 is shown when there is no patient as indicated in the system message area 440. The no sensor view 1130 is shown when a sensor is disconnected as indicated in the system message area 440. The no multiple parameter sensor view 1140 is shown when there is no multiple parameter sensor and the system message area 440 indicates searching for signal.

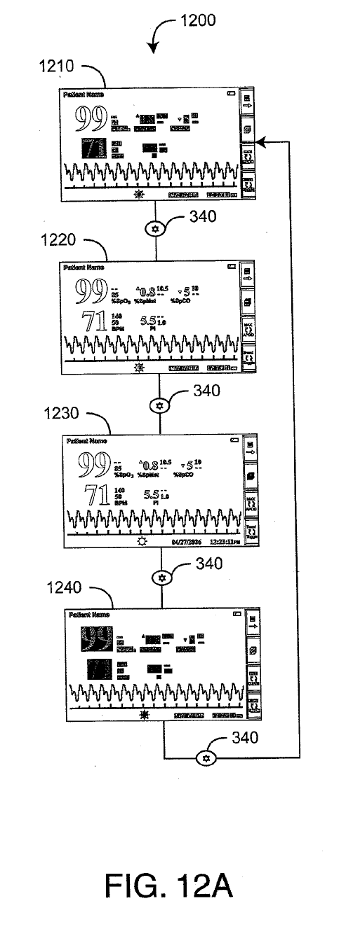

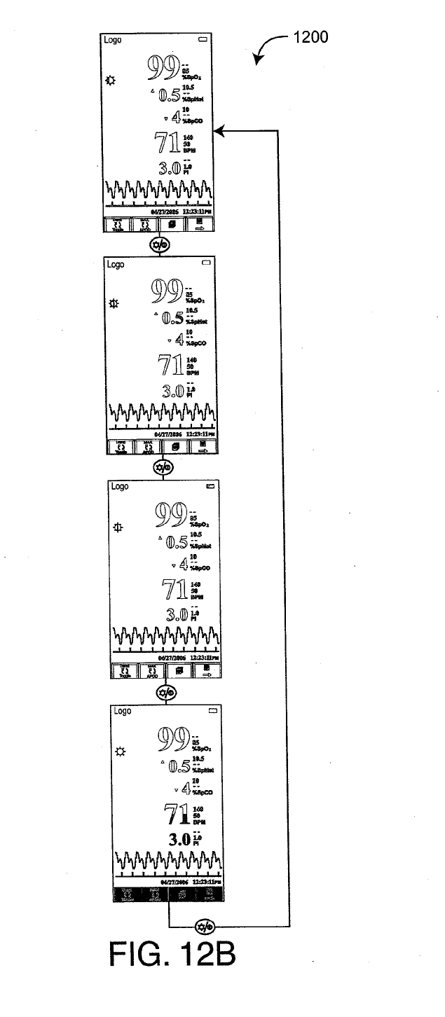

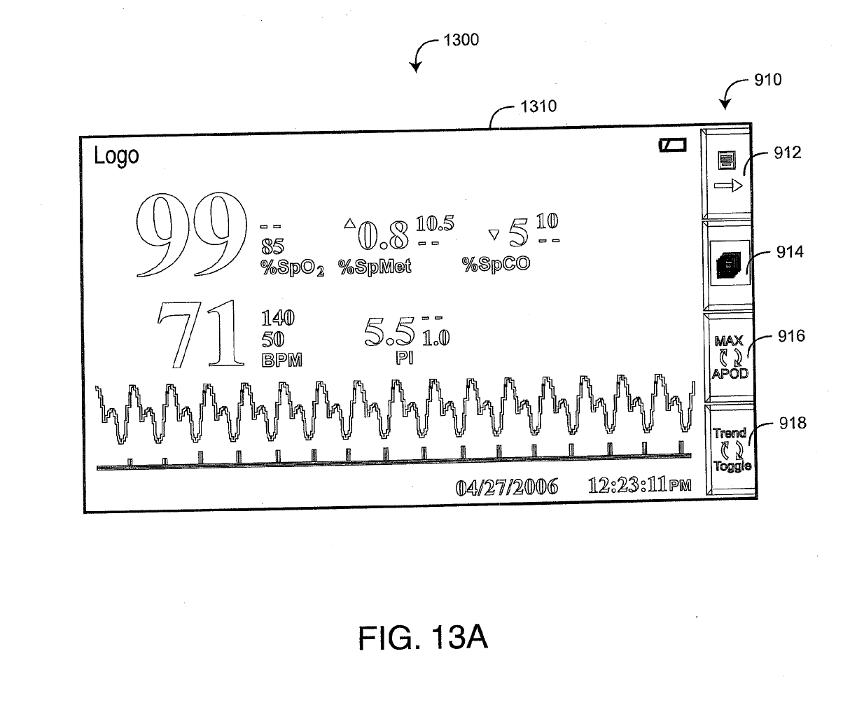

FIGS. 12A-B illustrate the contrast/brightness adjustment displays 1200 in horizontal and vertical formats. The displays 1200 include four levels of illumination 1210, 1220, 1230 and 1240. The illumination level of the backlight is changed by pressing the backlight button 340.

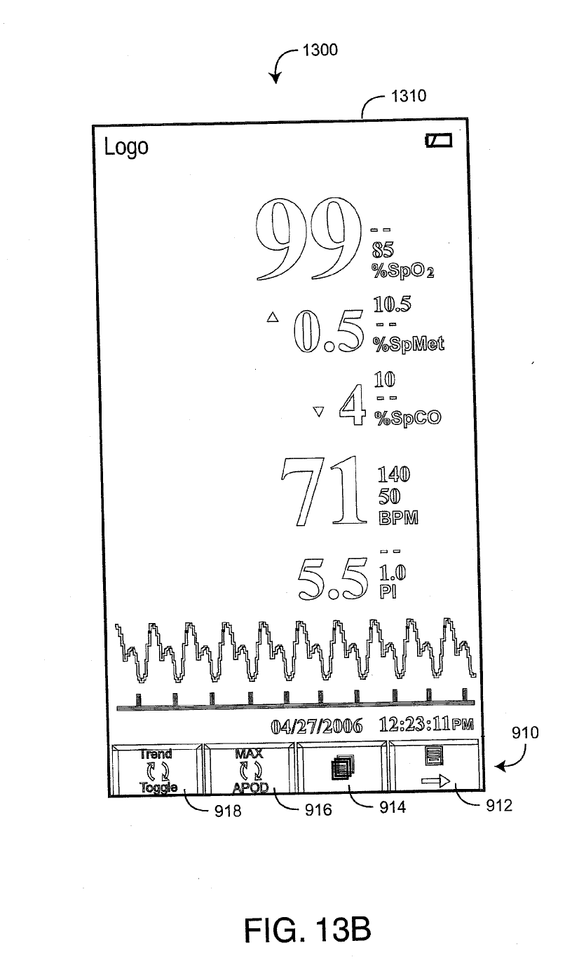

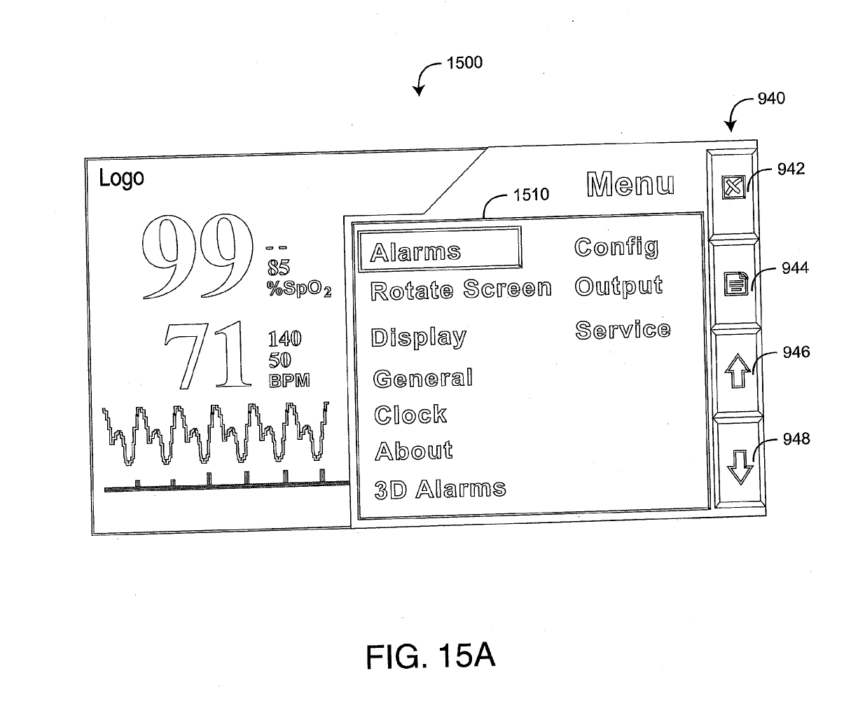

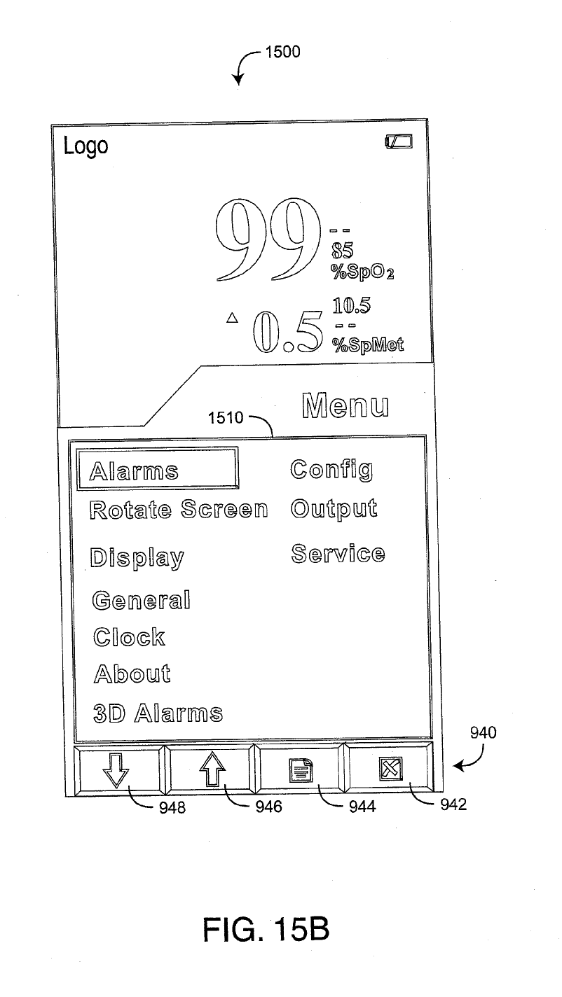

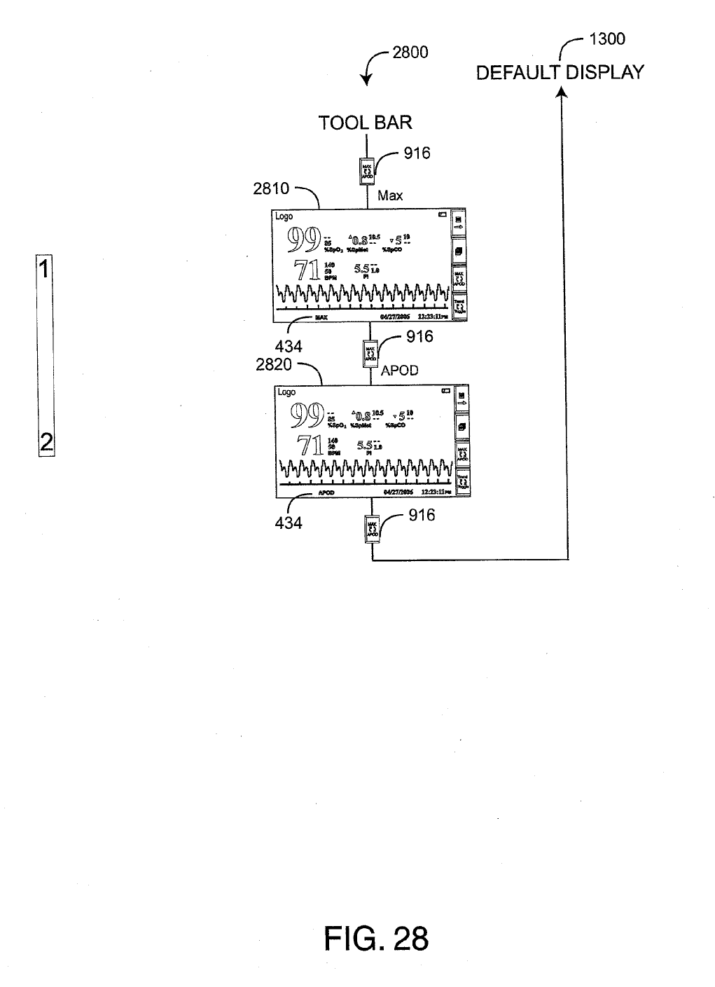

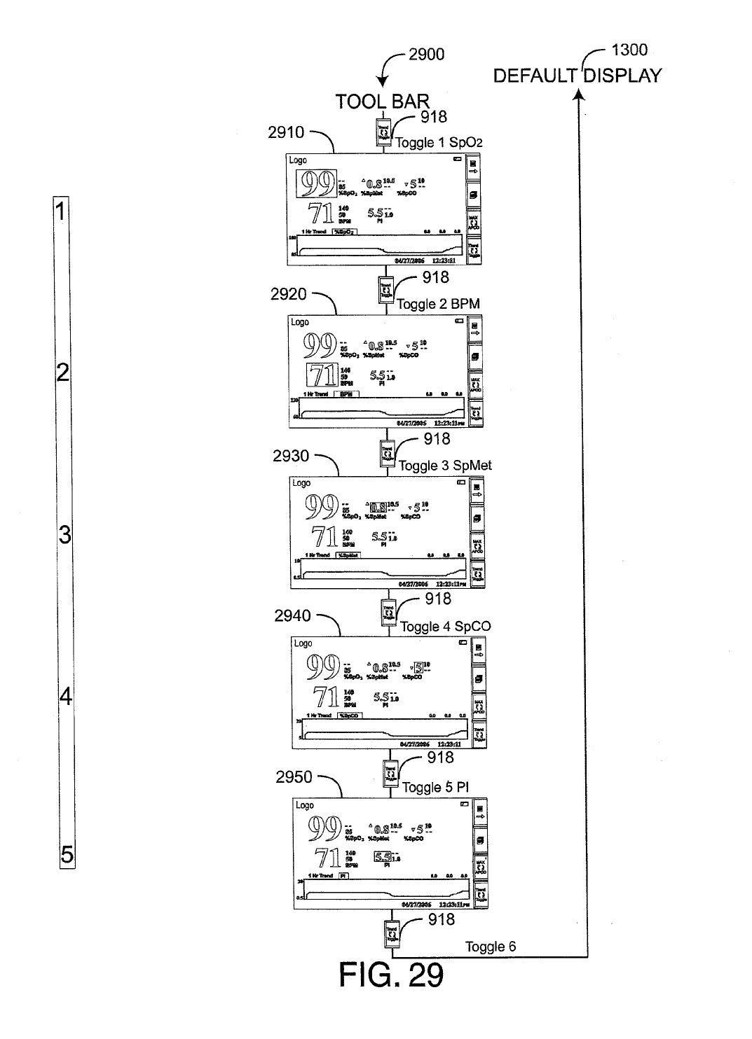

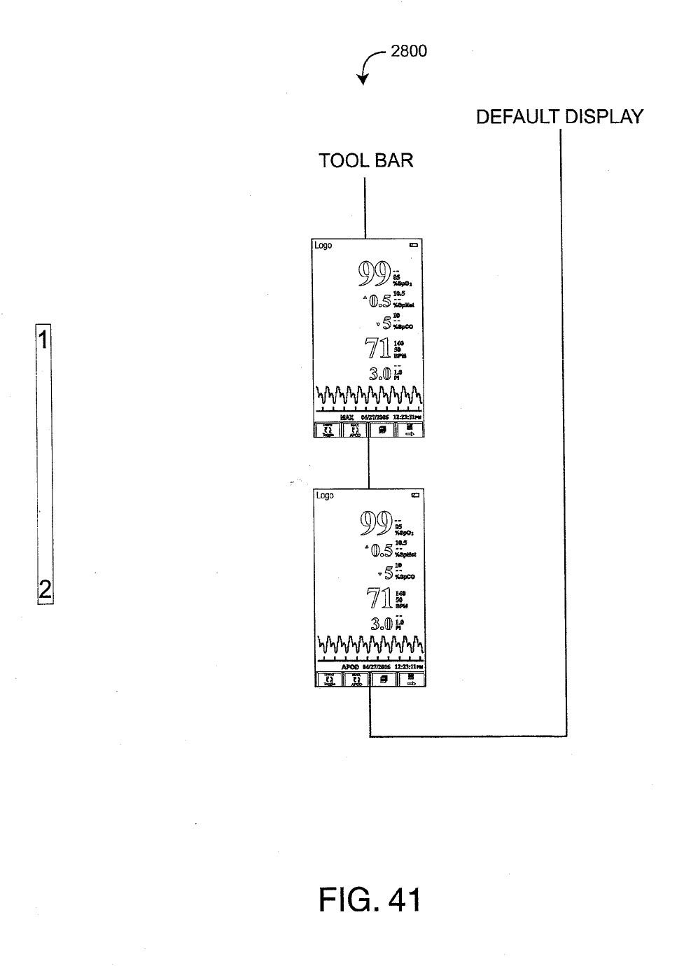

FIGS. 13A-B illustrate the default displays 1300 in horizontal and vertical formats. The default display 1300 has a pleth and signal IQ view 1310 and soft key icons 910 including a next menu page icon 912 selected to enter a tool bar display 1400 (FIGS. 14A-B), a menu icon 914 to enter a menu display 1500 (FIGS. 15A-B), a sensitivity icon 916 to enter a sensitivity display 2800 (FIGS. 28, 41) and a trend toggle icon 918 to enter a trend toggle display 2900 (FIG. 29).

FIGS. 14A-B illustrate tool bar displays 1400 in horizontal and vertical formats. The tool bar display 1400 has a next menu page icon 922 selected to enter a default display 1300 (FIGS. 13A-B), a trend display icon 924 to enter a trend display 1600 (FIG. 16), a volume up icon 926 and a volume down icon 928 to enter a volume adjustment display 1700 (FIGS. 17, 30).

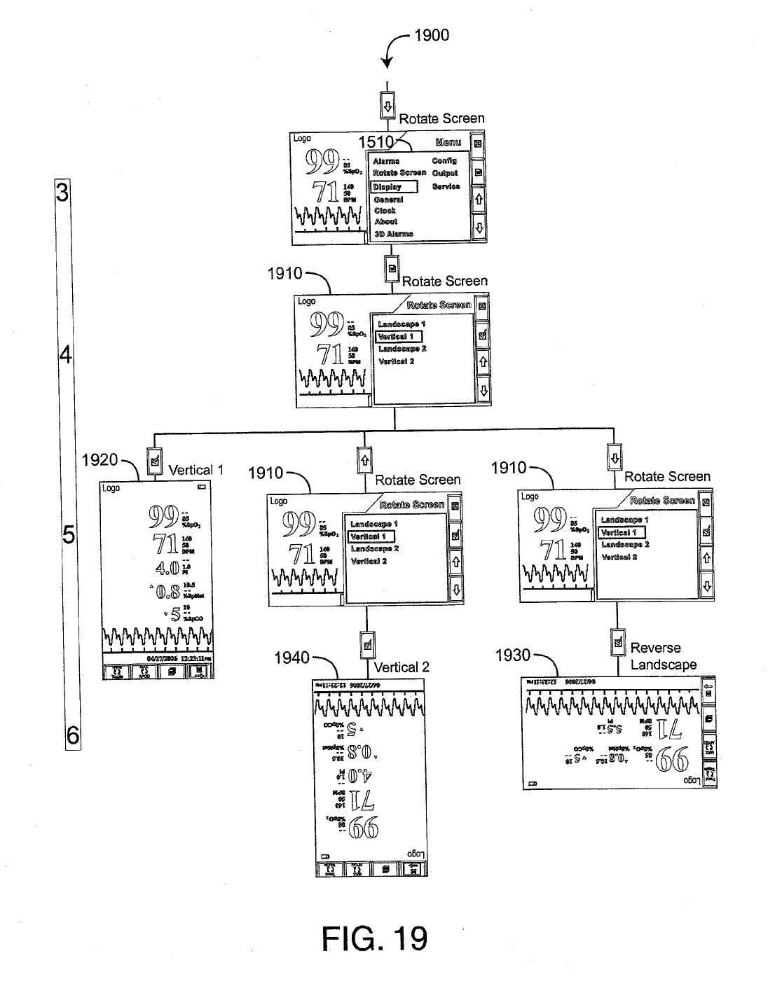

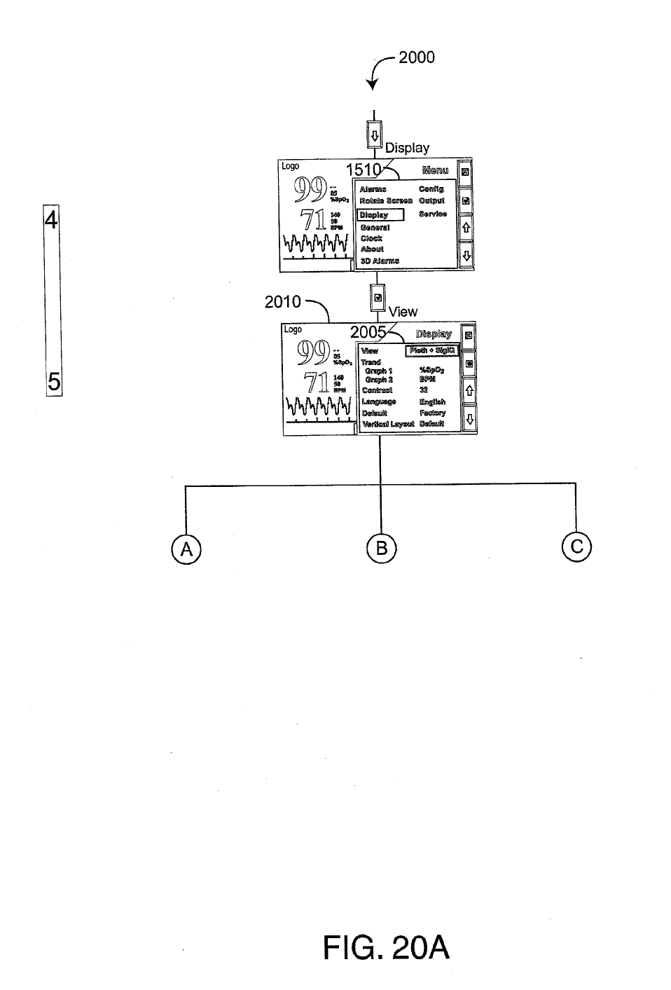

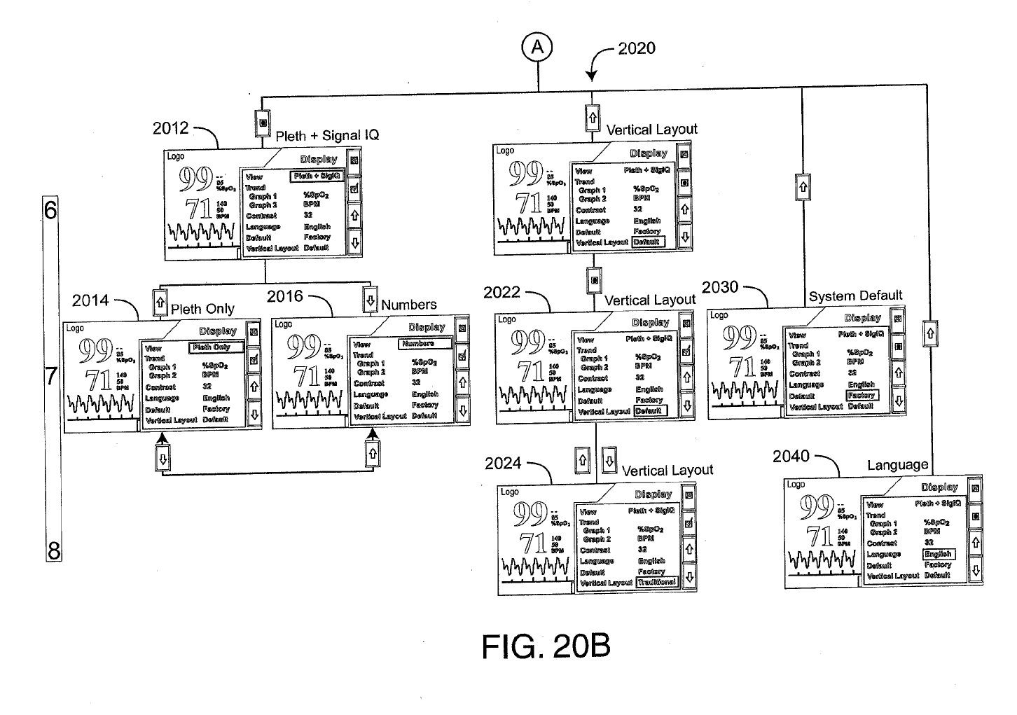

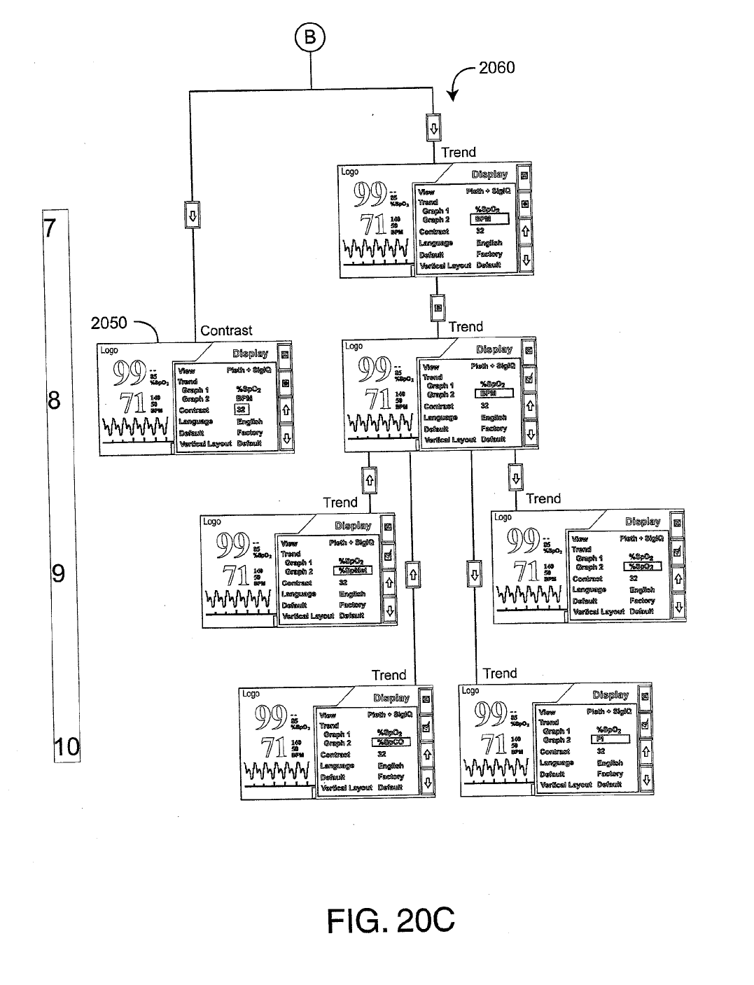

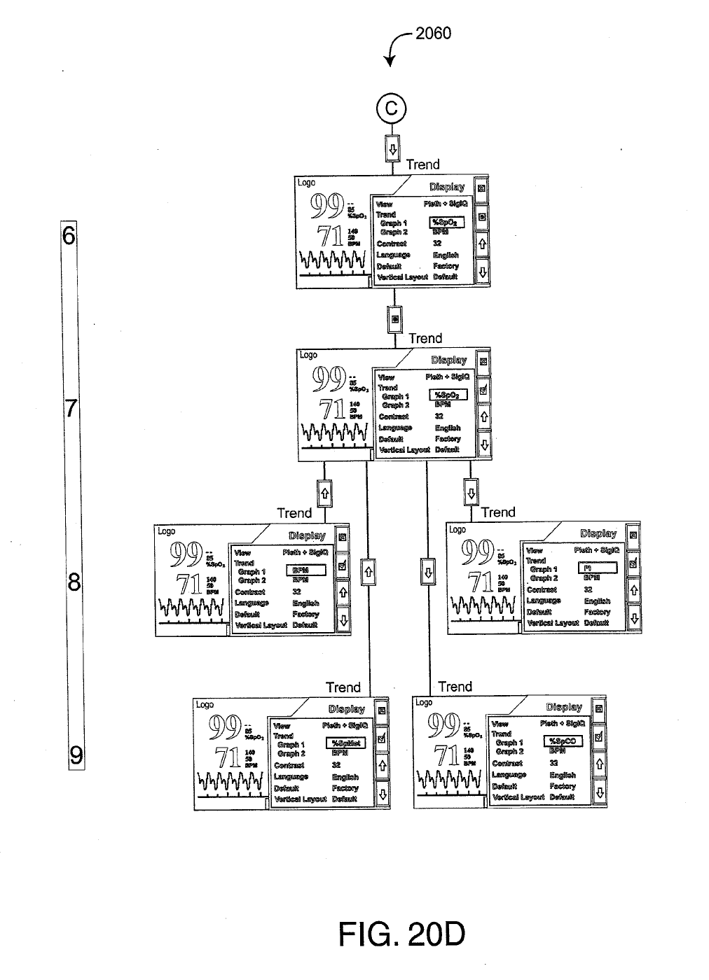

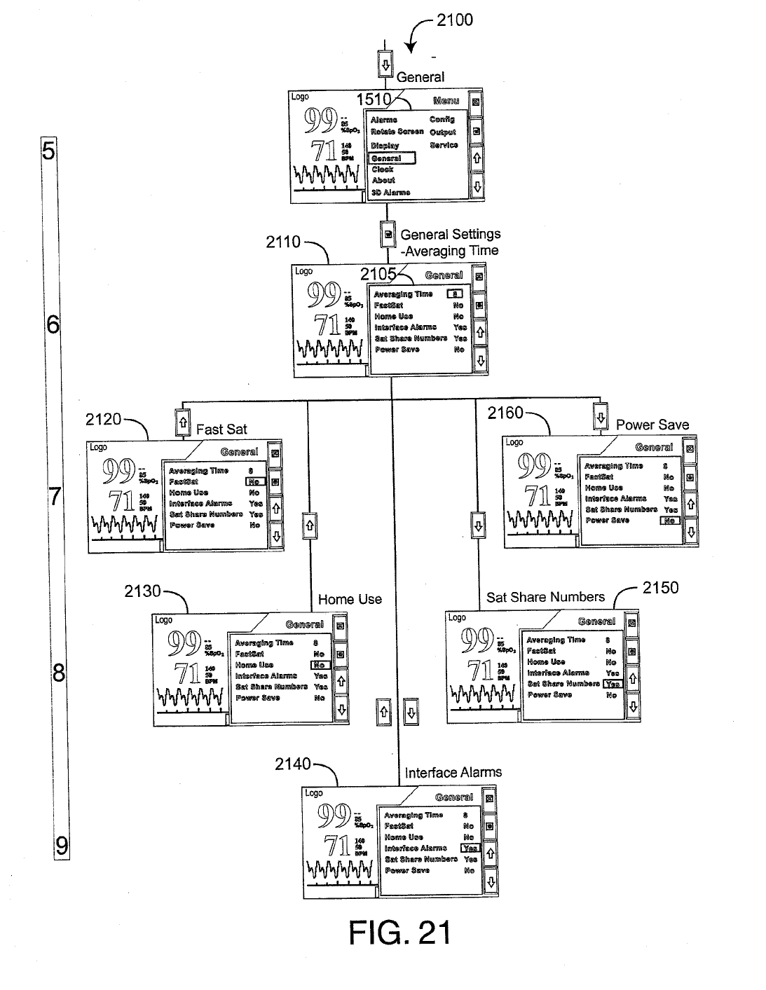

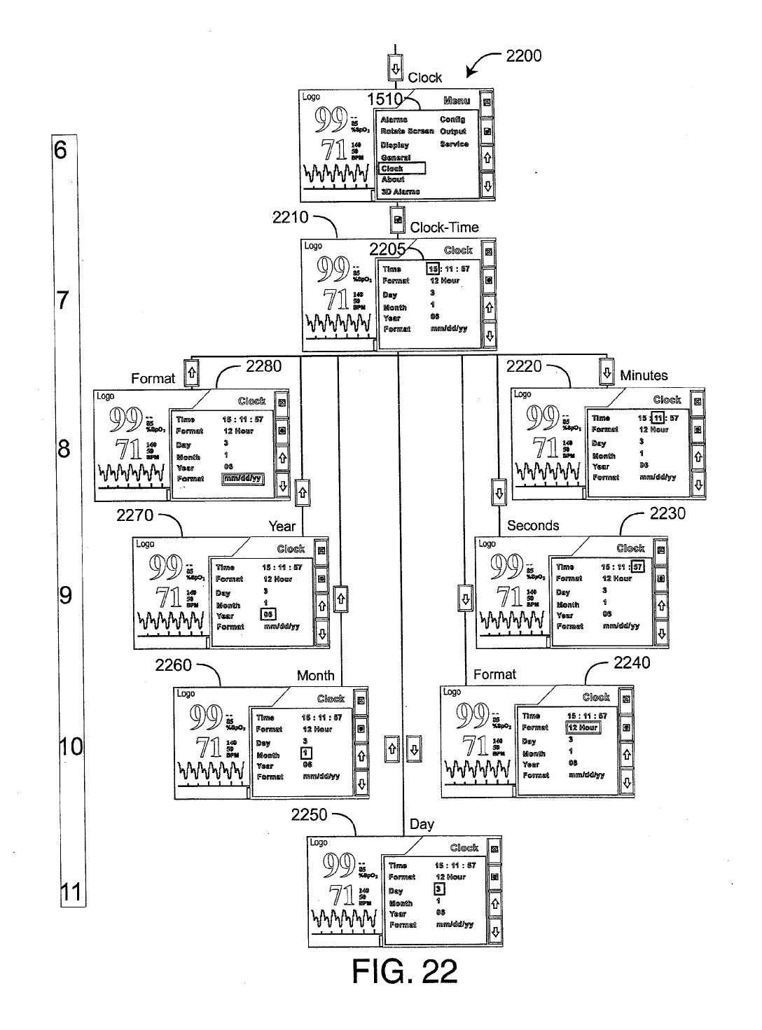

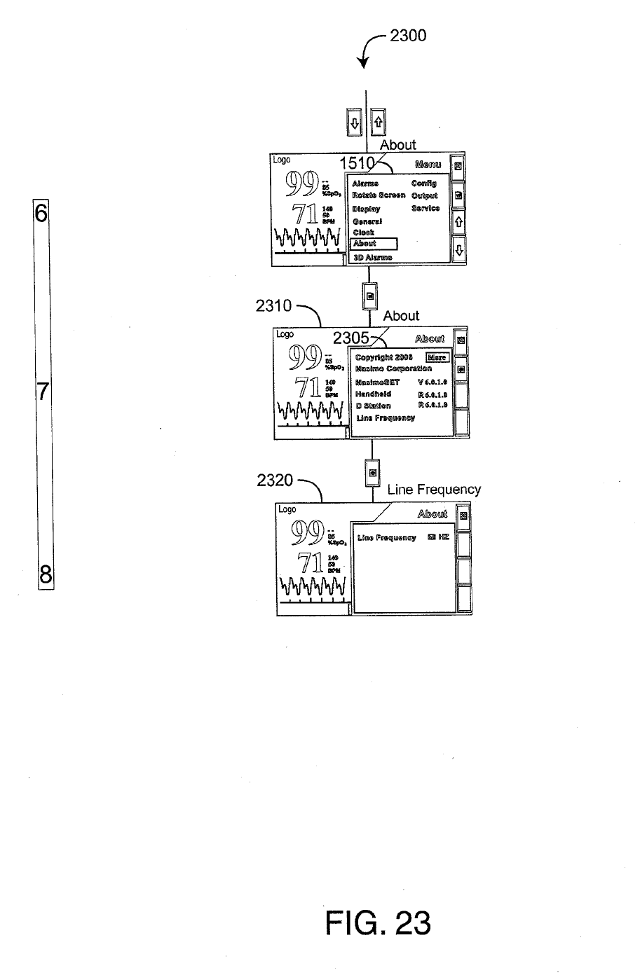

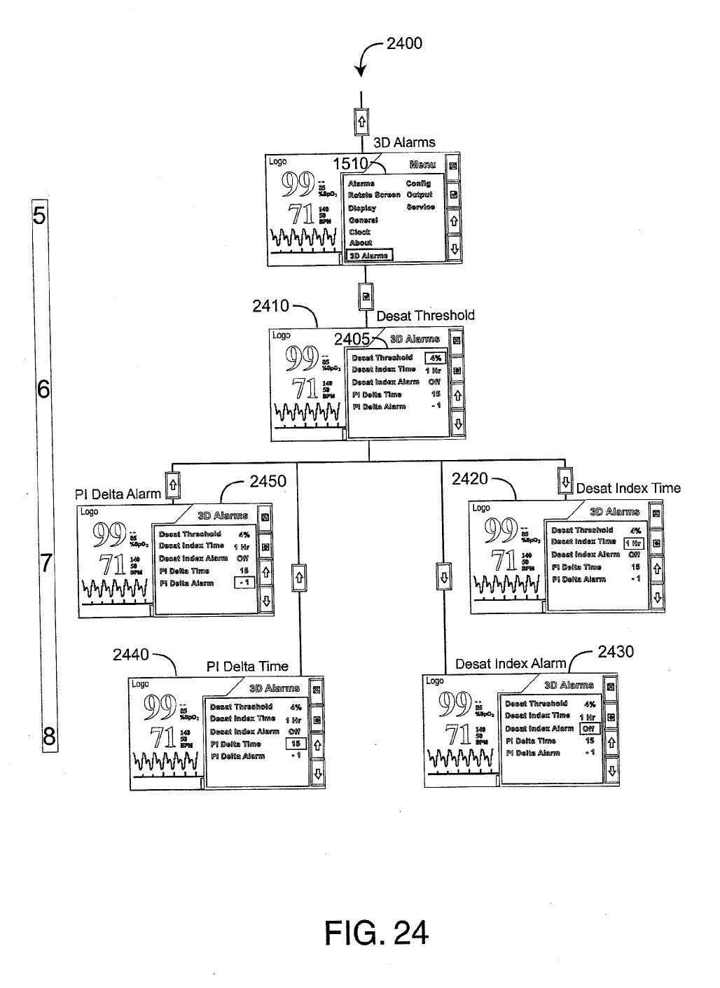

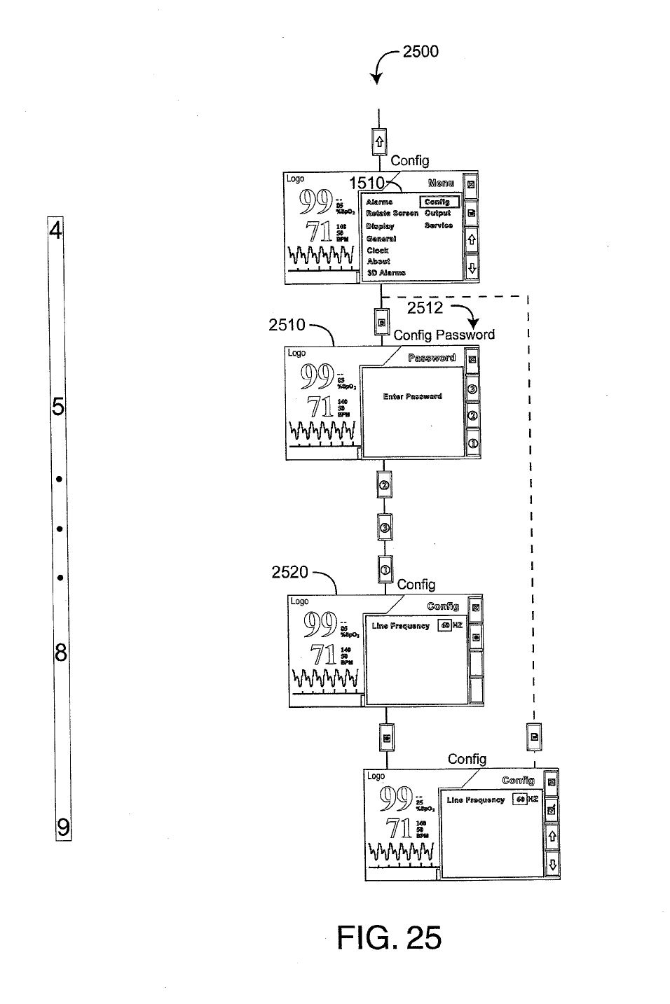

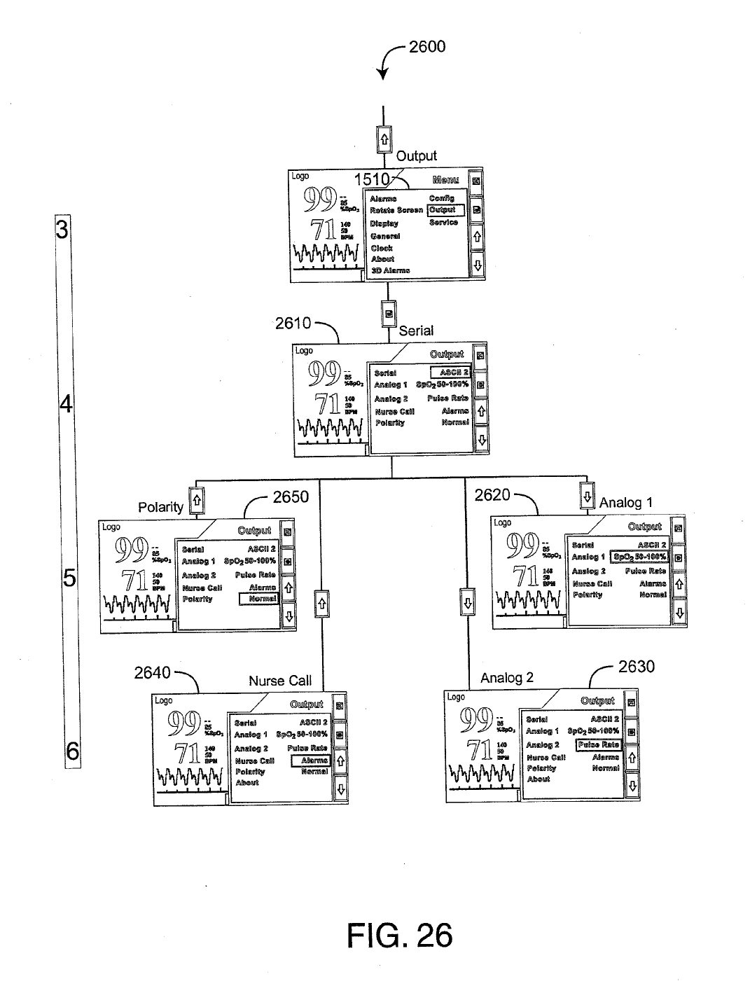

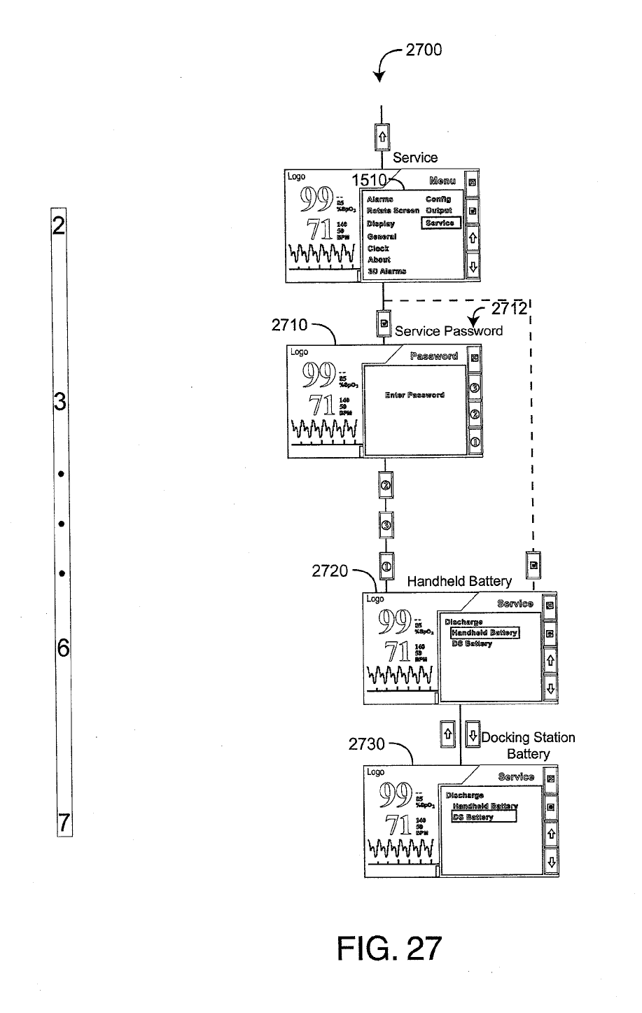

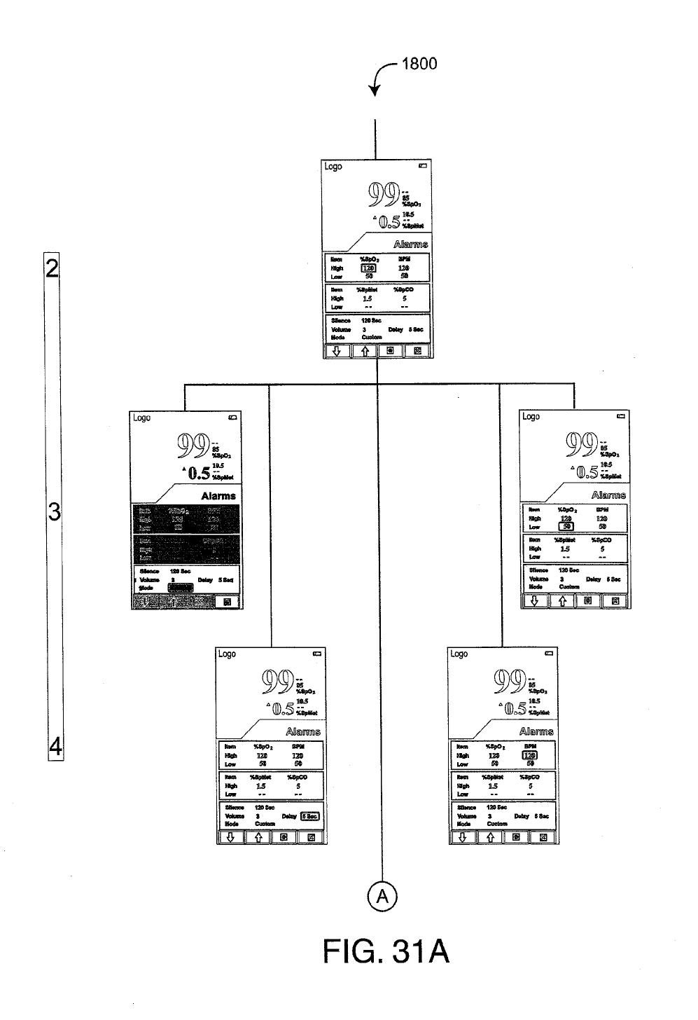

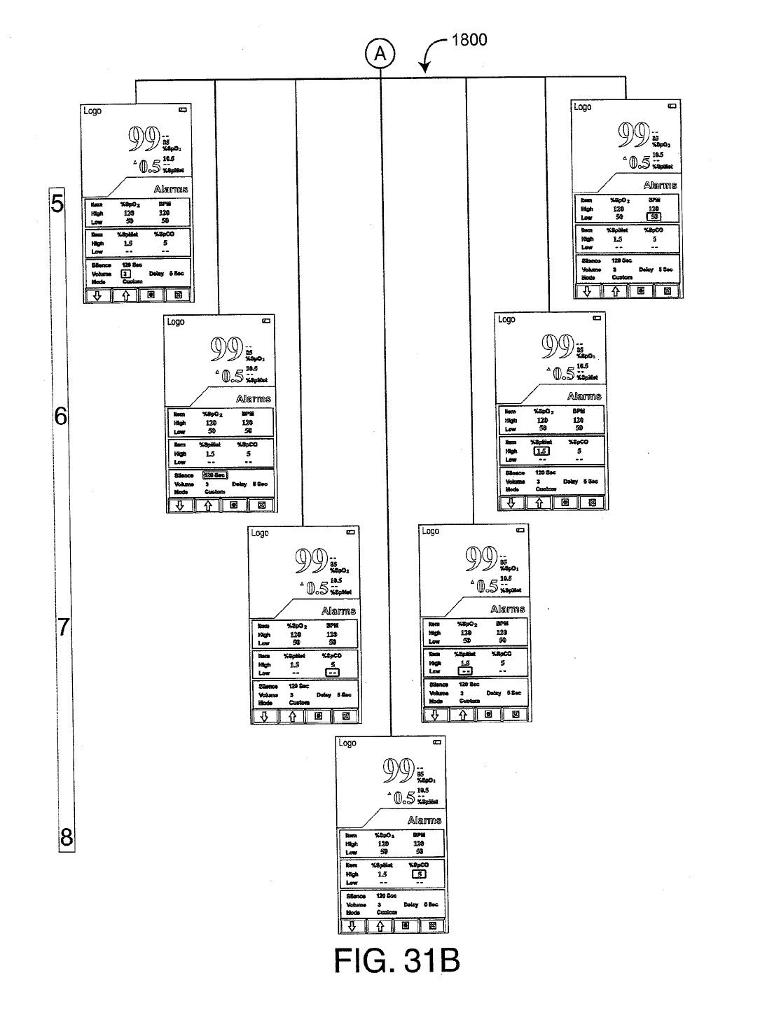

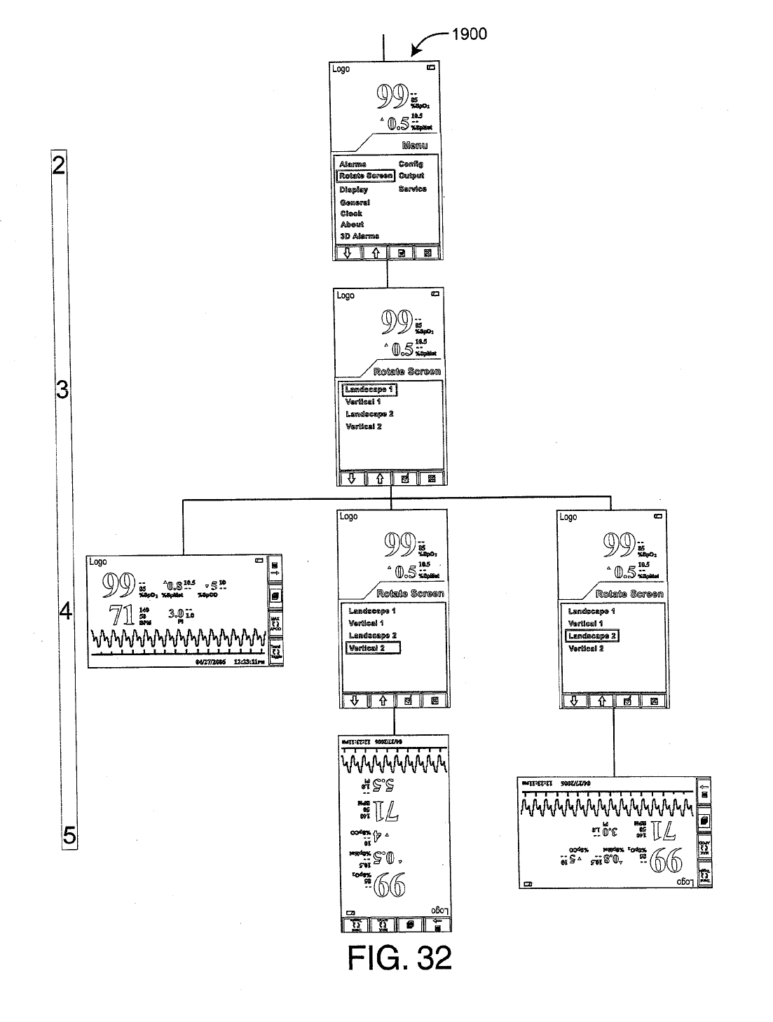

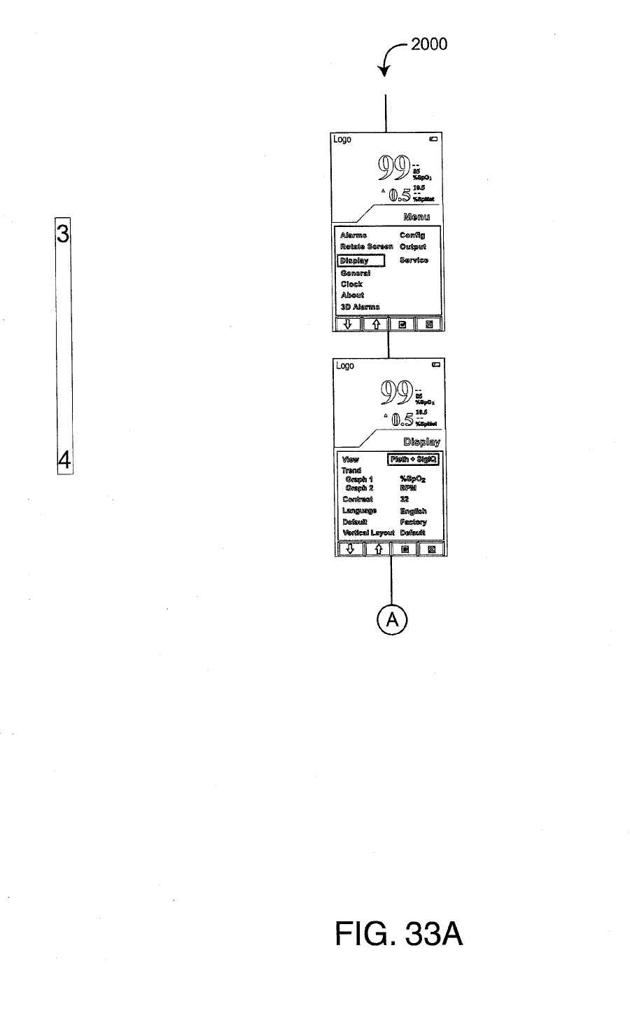

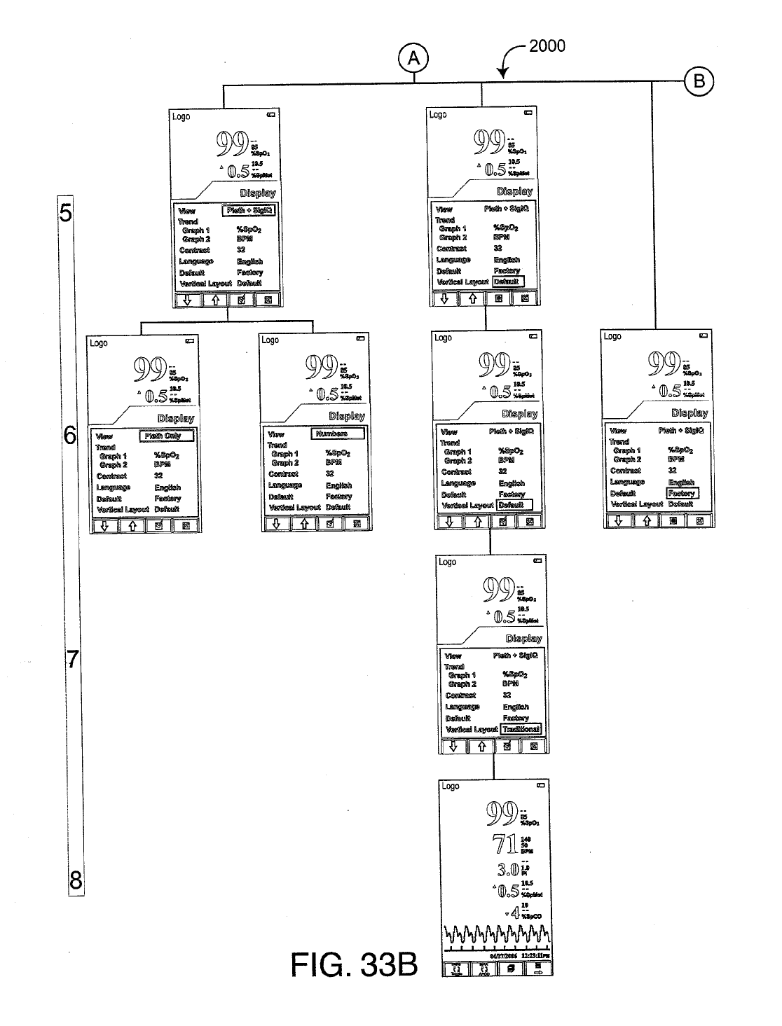

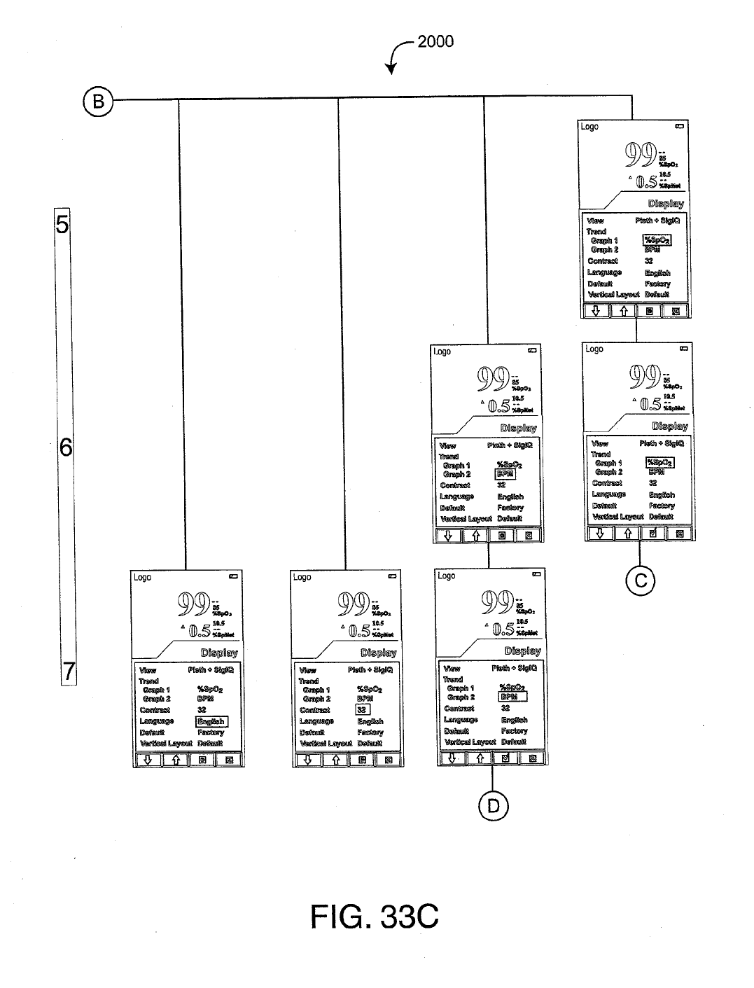

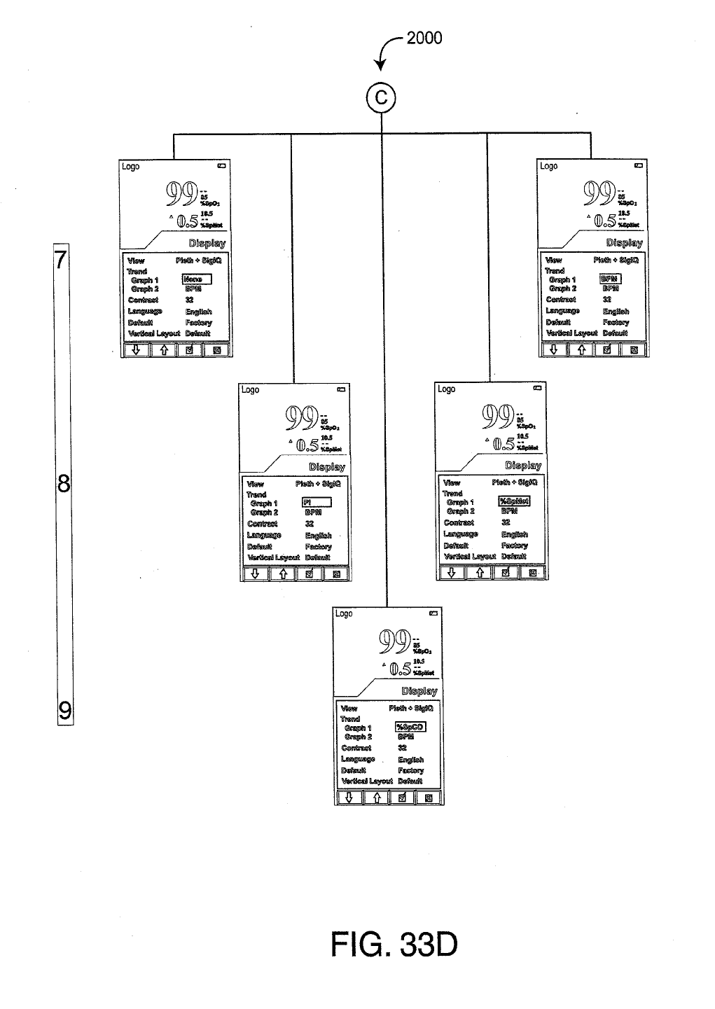

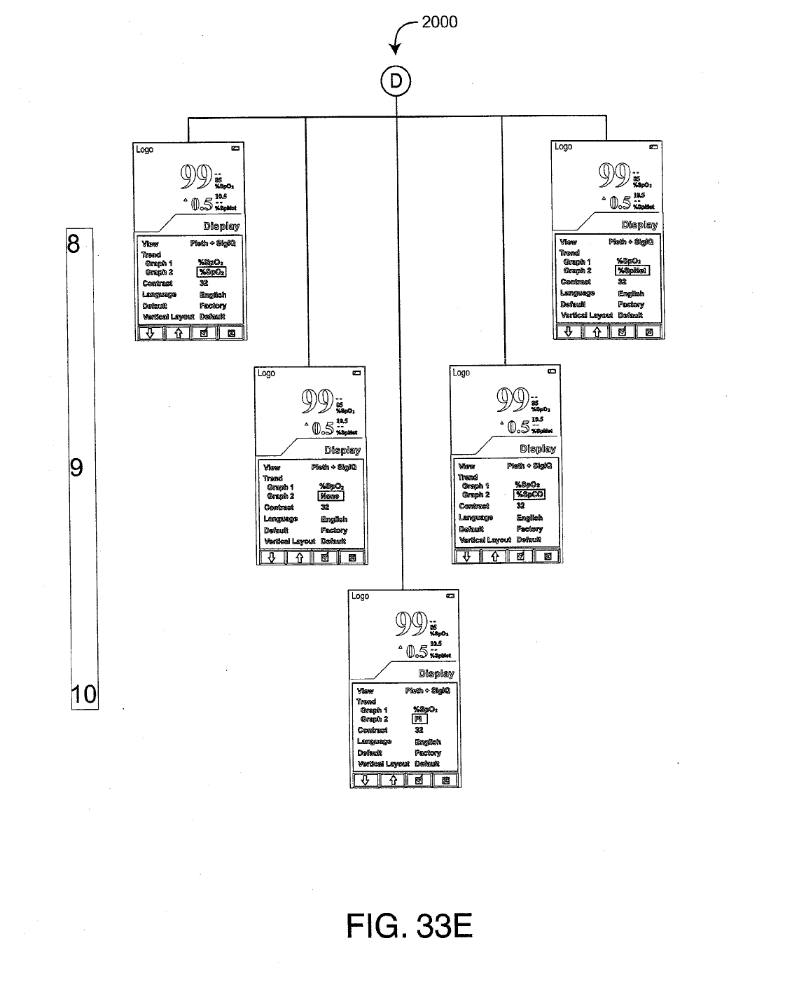

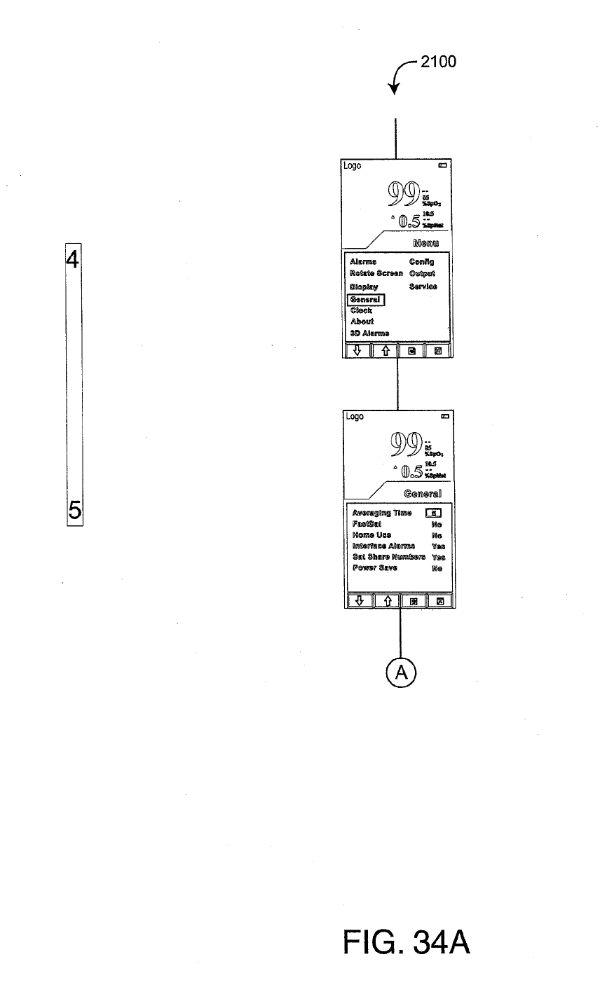

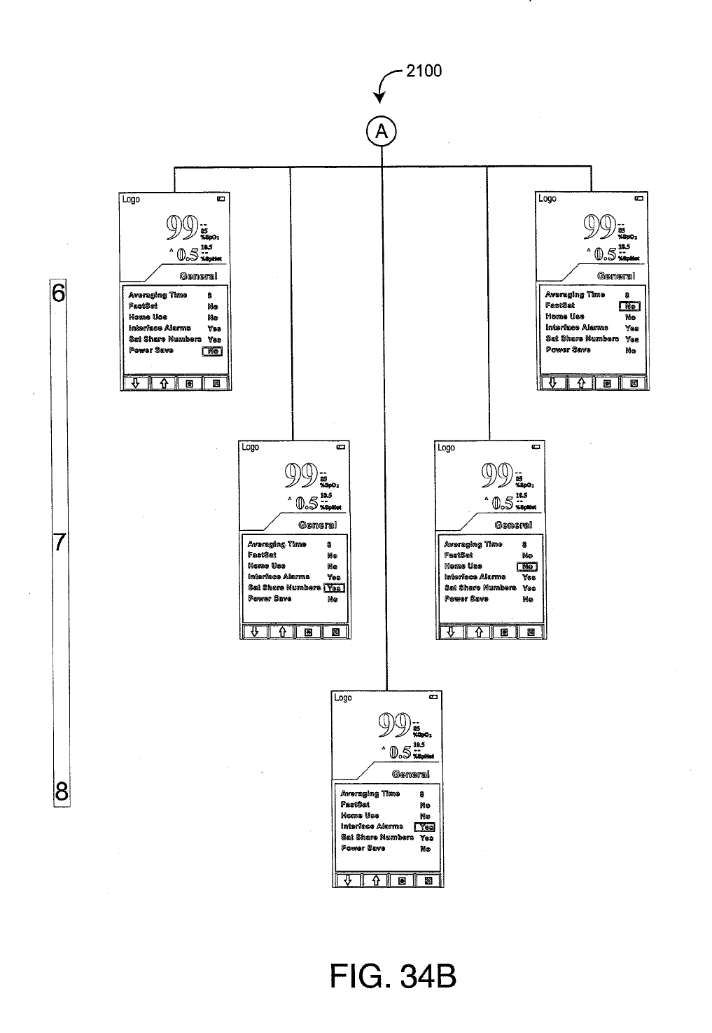

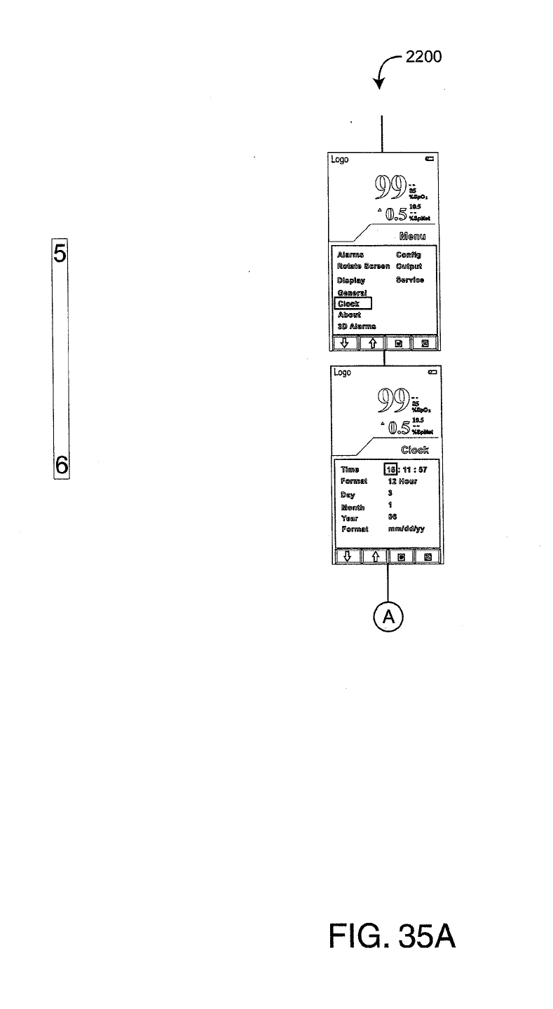

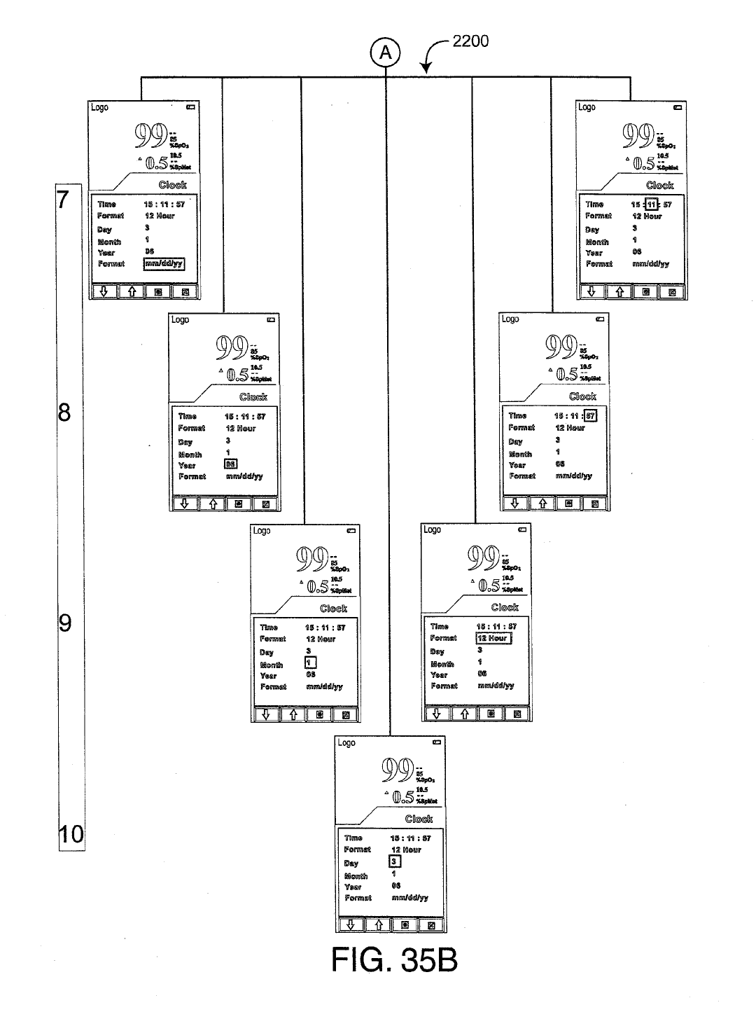

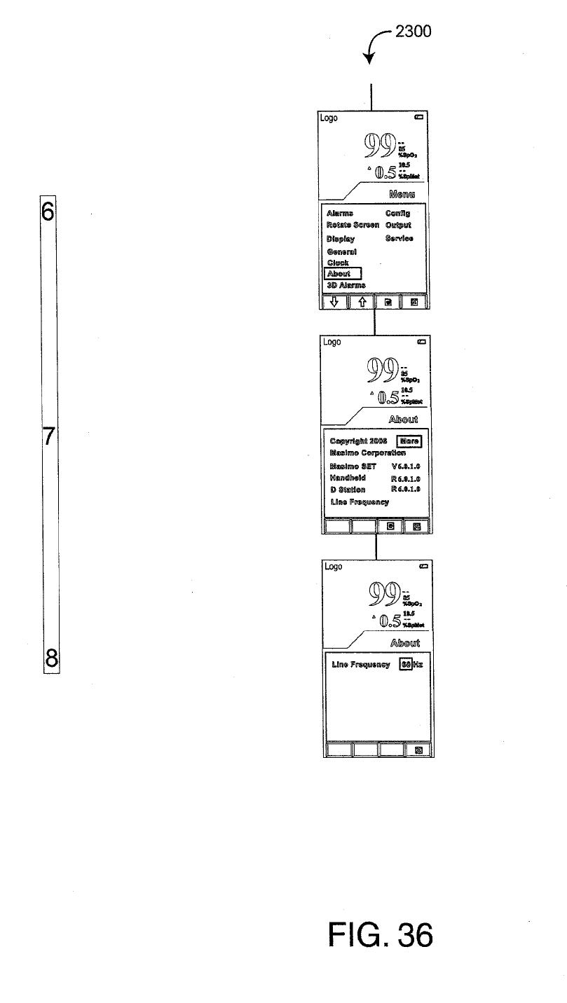

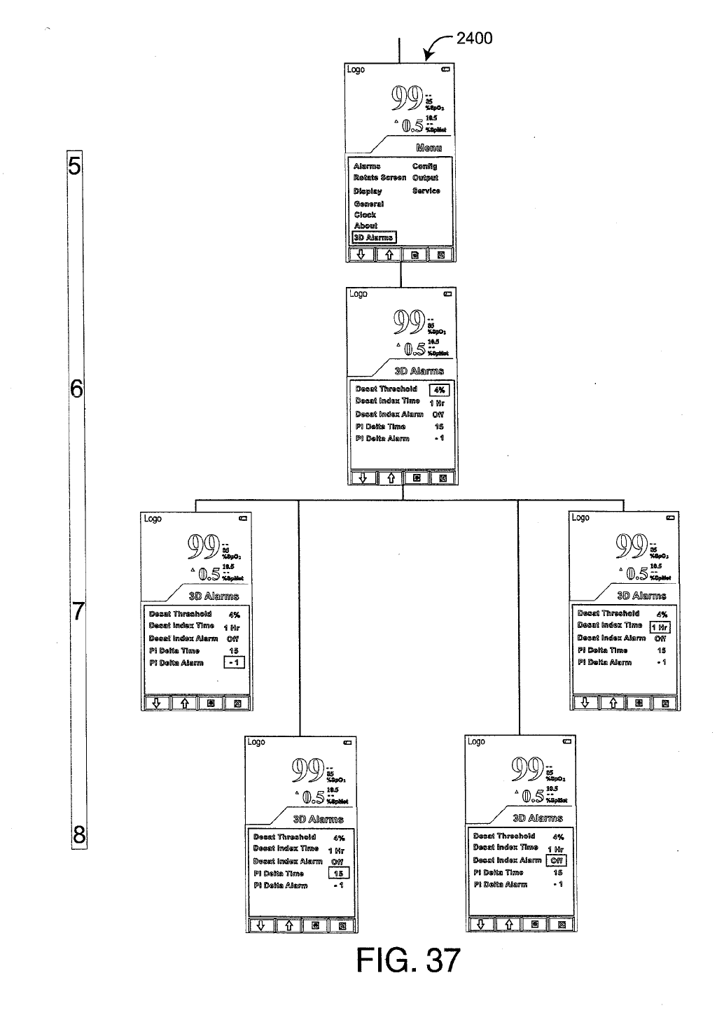

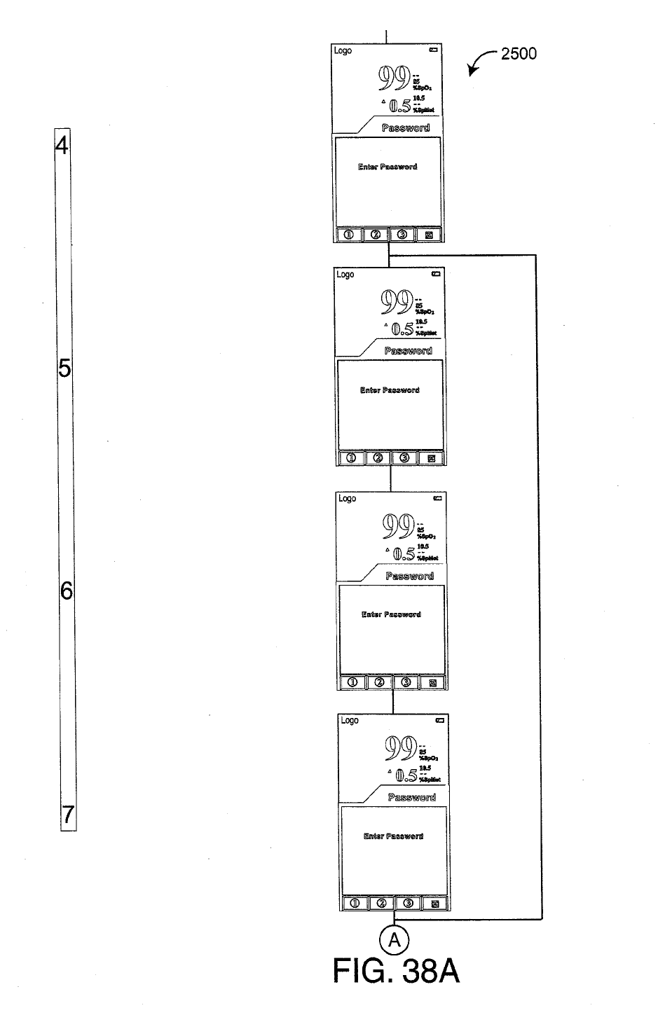

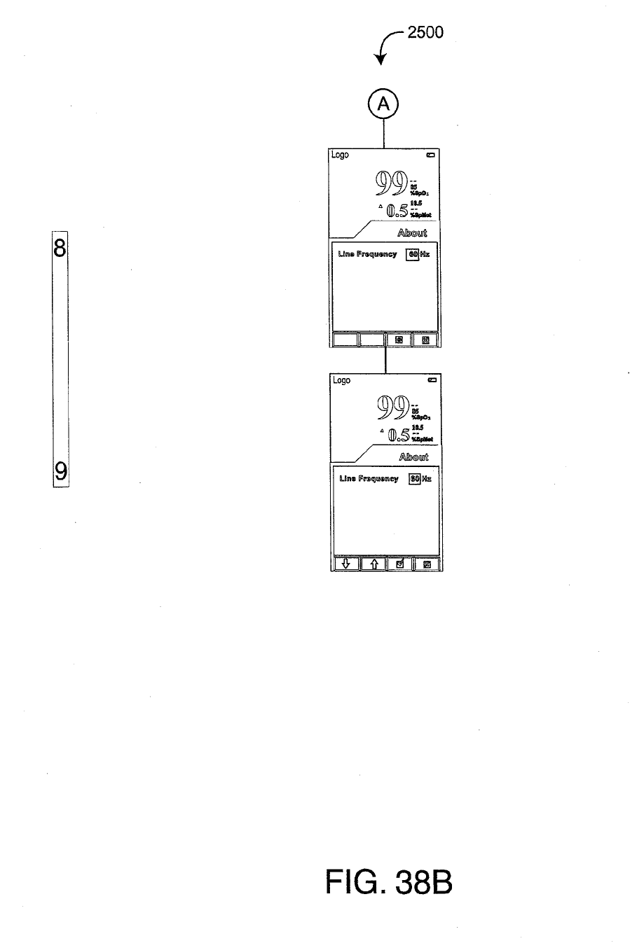

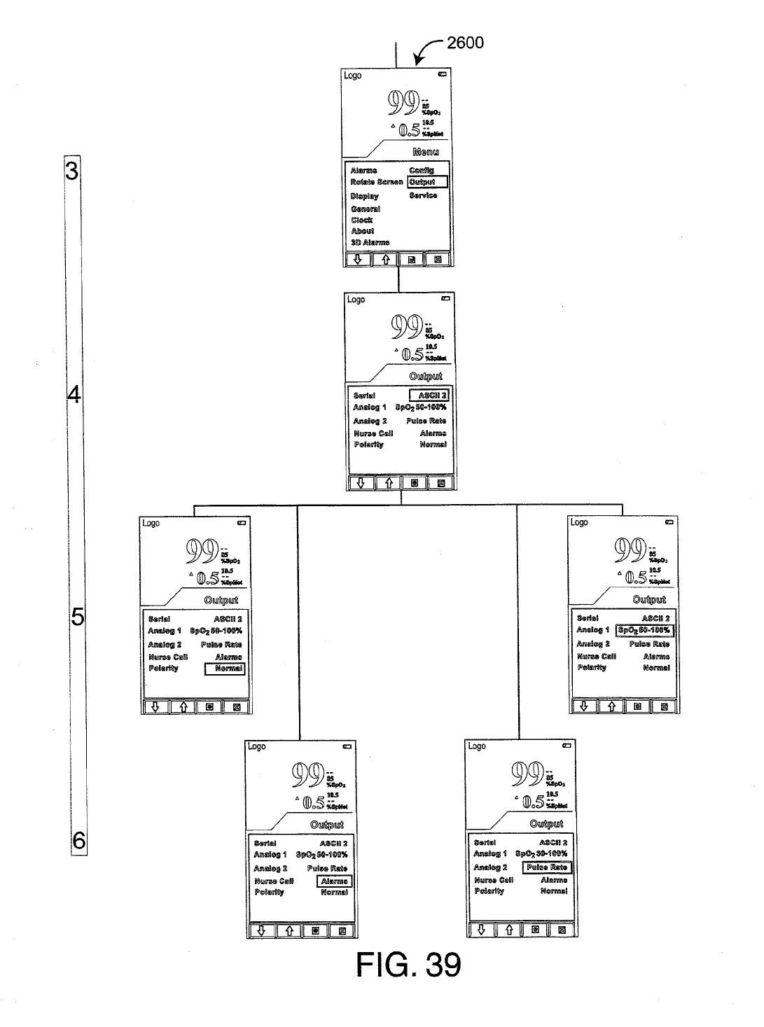

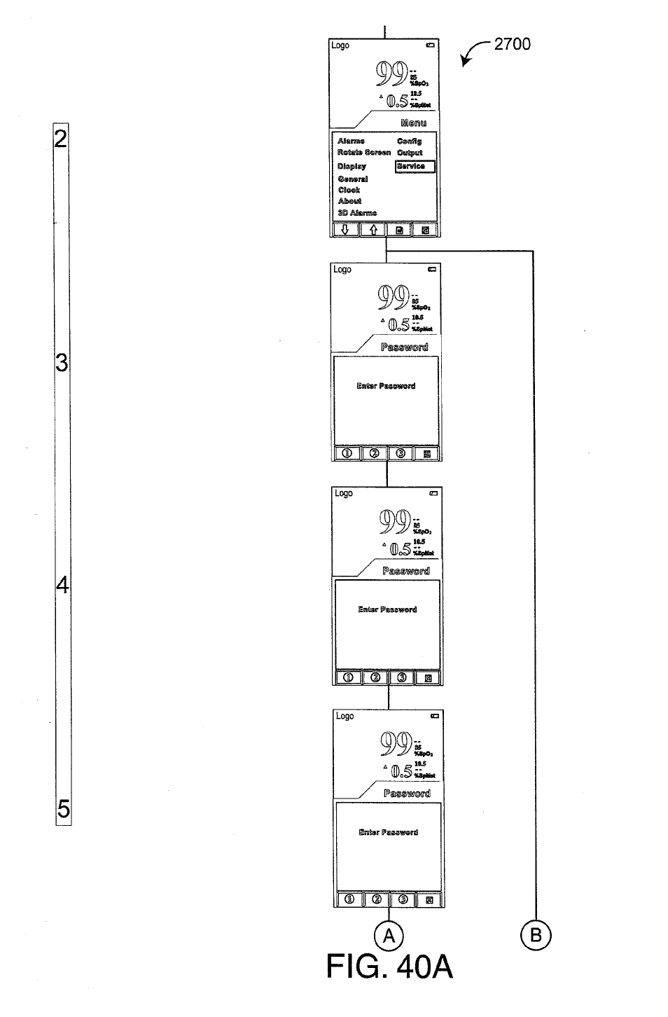

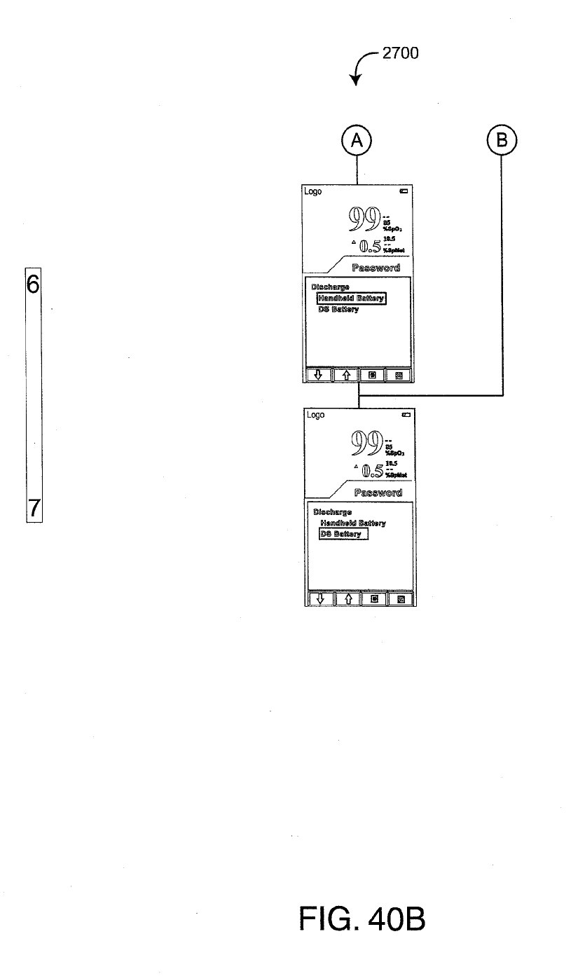

FIGS. 15A-B illustrate menu displays 1500 in horizontal and vertical formats. The menu display 1500 presents a display selection list 1510 associated with corresponding displays. The list 1510 includes alarms selected to enter an alarms display 1800 (FIGS. 18, 31), rotate screen to enter a rotate screen display 1900 (FIGS. 19, 32), display to enter a display set up display 2000 (FIGS. 20, 33), general to enter a general display 2100 (FIGS. 21, 34), clock to enter a clock display 2200 (FIGS. 22, 35), about to enter an about display 2300 (FIGS. 23, 36), 3D alarms to enter a 3D alarm system display 2400 (FIGS. 24, 37), config to enter a configuration display 2500 (FIGS. 25, 38), output to enter an output display 2600 (FIGS. 26, 39) and service to enter a service display 2700 (FIGS. 27, 40). The menu display 1500 has icons 940 for exit 942, select category 944, previous 946 and next 948, as described in detail with respect to FIG. 9C, above.

FIGS. 16A-B illustrate trend displays 1600. The monitor stores one data set of SpO₂, pulse rate, SpMet, SpCO, PI, PVI and system messages in a dedicated memory area. In one embodiment, the monitor can store between 72 hours and 18 days of trend data. The actual amount of trend data that is stored is dependent on the type of data that is collected. In one embodiment, the monitor only stores data in the trend memory while the device is turned on, and the trend data remains in memory until the memory fills up, or is cleared by the user. For example, trend memory capacity is a minimum of 72 hours for a trend period of 2 seconds. Trend memory capacity is typically 435 hours for a trend period of 10 seconds. In one embodiment, a trend display is configured to display one or two of any of the six available trend parameters of SpO₂, SpMet, SpCO, pulse rate, PI or PVI that are selected by a user. The unit is storing all 6 parameters in trend memory, but can only display one or two user selected parameters at any one time. The trend display can be adjusted to the desired parameter by selecting the trend display icon 924 from the tool bar display 1400 (FIGS. 14A-B).

As shown in FIG. 16A, once the trend display icon 924 is selected, the trend data are displayed on the main screen as a trend display 1610. The trend display 1610 has a top line 1612, a second line 1614 and a trend graph 1616. The top line 1612 shows the time scale of the trend graph, followed by the starting date, starting time and end time of the data set that is displayed on the screen. The second line 1614 shows the minimum, maximum and average SpO₂, HbMet, HbCO, PI, pulse rate or PVI measurements contained in the displayed data set, excluding zero measurements. The trend graph 1616 shows the desired trend parameter measurement displayed versus time. In one embodiment, the trend display 1610 has two trend graphs showing two of the desired trend parameter measurements. A vertical line on the trend graph 1616 indicates the averaged data, while a horizontal line shows beginning and ending periods of the trend or when the sensor was removed from the patient. The trend display 1610 also has the first page of soft key icons 970. When next menu page 972 is selected, a trend toolbar B display 1620 is entered. The trend toolbar B display 1620 includes the second page of soft key icons 980 and the same view as that of the trend display 1610. When next menu page 982 is selected, a trend toolbar C display 1630 is entered including the third page of soft key icons 990 and the same view as that of the trend display 1610. The first page soft key icons 970, second page soft key icons 980 and third page soft key icons 990 are described in detail with respect to FIG. 9D, above.

Also as shown in FIG. 16A, once trend setup 994 from the third page soft key icons 990 is selected, a trend setup menu 1640 is shown that allows a user to set the default trend settings or download the trend data. The default settings are used to scale the trend graphs when the trend data button located on the main display is accessed. As shown in FIGS. 16A-B, the trend setup menu 1640 is capable of setting up maximum and minimum scales of SpO₂, HbMet, HbCO, PI, pulse rate and PVI graphs, default view, trend action and period by selecting previous icons 1642 and next icons 1644. The default view selects the default time scale of the trend view. This setting only selects the time scale of the trend view when the trend data is initially displayed, i.e. when the trend data is initially accessed.

In one embodiment, the selections are 24 hrs, 12 hrs, 8 hrs, 4 hrs, 2 hrs, 1 hr, 30 minutes, 10 minutes, 1 minute and 20 seconds. The trend action has options of serial dump, analog dump and print. The serial dump option is selected to send all the data that is stored in trend memory to the serial port. This option is used to communicate the stored data set to trend graphing software applications. The analog dump option is selected to send all the data that is stored in the trend memory to the analog output. This option is used to print the trend information on an analog chart recorder. The print option is selected to print the trend data that is shown in the trend view. The trend data is first printed in histogram format, followed by a table of data that shows the time and date stamp of a trend record, and the SpO₂, pulse rate, HbMet, HbCO and PI measurement. Each trend record is printed on a single line followed by a carriage return and line feed character. The Trend period setting determines how often a set of SpO₂, pulse rate, HbMet, HbCO and PI data points is stored in trend memory. A setting of 2, for example, sets the monitor to store one set of SpO₂, pulse rate, HbMet, HbCO and PI measurements every 2 seconds, resulting in a minimum trend capacity of 72 hours. A setting of 10, for example sets the monitor to store one set of data points every 10 seconds, resulting in a typical trend storage capacity of 18 days.

Additionally shown in FIG. 16A, once histogram 996 is selected, a histogram display 1650 is shown to display the trend data of selected parameters as numerical percentages shown in specific ranges. Once clear data 998 is selected, a clear data display 1660 is shown requiring a confirmation of the operation that will clear the data stored in the trend memory.

FIGS. 17 and 30 illustrate volume adjustment displays 1700 to adjust the volume of the pulse beep by selecting an increase loudness icon 926 and a decrease loudness icon 928. In one embodiment, seven levels of volume exist.

FIGS. 18A-B and 31A-B illustrate alarms displays 1800 that are shown once the select category icon 944 is selected and “alarms” is selected in the menu selection list 1510 (FIGS. 15A-B). Alarm limits are checked each time the monitor is used to ensure that they are appropriate for the patient being monitored. An audible alarm and a flashing alarm icon (and indicator light) occur when an alarm limit is exceeded. The alarms displays 1800 include a SpO₂ high limit display 1810, a SpO₂ low limit display 1815, a BPM high limit display 1820, a BPM low limit display 1825, a HbMet high limit display 1830, a HbMet low limit display 1835, a HbCO high limit display 1840, a HbCO low limit display 1845, a silence display 1850, a volume display 1860, a delay display 1865 and a mode display 1870. The displays 1810-1870 are shown by selecting previous icons 956 and next icons 958.

As shown in FIGS. 18A-B, the displays 1810-1845 allow a user to set high and low limits of SpO₂, BPM, HbMet and HbCO. In one embodiment, the SpO₂ high alarm limit can be set anywhere between 2% and 99%, with a 1% step size. In the off setting, the alarm can be turned off completely. The SpO₂ low alarm limit can be set anywhere between 1% and 99%, with a 1% step size. The SpO₂ low limit can not be set below the password protected minimum low SpO₂ alarm limit. The pulse rate high alarm limit can be set anywhere between 30 BPM and 240 BPM, with a 5 BPM step size. The pulse rate low alarm limit can be set anywhere between 25 BPM and 235 BPM, with a 5 BPM step size. The HbMet high alarm limit can be set anywhere between 1% to 100%. Between 1% and 2%, the step increment is 0.1%. Between 2% and 100%, the step increment is 0.5%. The HbMet low alarm limit can be set anywhere between 0.1% to 99.5%. Between 0.1% and 2%, the step increment is 0.1%. Between 2% and 99.5%, the step increment is 0.5%. In the off setting, the alarm can be turned off completely. The HbCO high alarm limit can be set anywhere between 2% and 100%, with a 1% step size. The HbCO low alarm limit can be set anywhere between 1% and 99%, with a 1% step size. In the off setting, the alarm can be turned off completely. The low alarm limit always has to be set below the high alarm setting. When the high alarm limit is set below the low alarm limit, the low alarm limit will automatically adjust to the next setting below the newly entered high alarm limit setting.

Also shown in FIGS. 18A-B, the silence display 1850 allows a user to set the alarm silence period. An alarm is silenced by pressing the alarm silence button on the front panel. The alarm silence can be set for the durations of 30, 60, 90, 120 seconds. As an indicator that the alarm system is silenced, the alarm status indicator 419 (FIG. 4A) is shown as a bell with a slash through it. A timer is shown next to the bell indicating the remaining alarm silence duration. In a particular embodiment, the alarm silence period is reset to 120 seconds or 90 seconds in neonatal mode upon power cycle. The alarm silence can also be set to all mute so that all patient alarm conditions are silenced. Only system alarms will be indicated by an audible alarm. As an indicator that the system is set to all mute, the alarm status indicator 419 (FIG. 4A) is shown as a bell with a slash through it. The alarm silence can further be set to all mute with an audible reminder. As a reminder, a single audible alarm will occur every three minutes. If an alarm condition occurs while the alarm silence period is set to all mute, the only alarm indications will be visual displays and symbols related to the alarm condition. No alarm tone will sound.

Further shown in FIGS. 18A-B, the volume display 1860 allows a user to set the alarm volume. In one embodiment, four levels are available that level 1 is the softest and level 4 is the loudest. The device retains the alarm volume setting upon a power cycle. The delay display 1865 allows a user to set an audible saturation delay. The delay can be set to either of 0, 5 or 10 seconds. The delay setting only affects saturation alarm indications. In custom mode, the unit will retain the alarm delay setting after a power cycle. In neo or adult mode the unit will reset the alarm delay to the hospital specified setting after a power cycle.

Additionally shown in FIGS. 18A-B, the mode display 1870 allows a use to set mode limits. In one embodiment, three types of modes including adult, neonatal and custom are available. Adult and neonatal modes must be initially set and enabled via password protected screen before they can be selected. For a custom mode, any changes to settings on the alarm menu will be retained after a power cycle. Once custom values are changed by the user, they will be retained after a power cycle. For an adult mode, any changes to settings on the alarm menu will be reset to pre-defined adult defaults after a power cycle. For a neo mode, any changes to settings on the alarm menu will be reset to predefined neonatal defaults after a power cycle. Adult and neo settings can be changed via a password protected screen, to specific hospital requirements. If the settings are changed, any values are changed by the user will be returned to the unit's default values after it is powered down.

FIGS. 19 and 32 illustrate rotate screen displays 1900. Once “rotate screen” in the menu selection list 1510 is selected, a rotate screen 1910 is displayed. The rotate screen 1910 has a selection list 1912 of landscape 1, vertical 1, landscape 2 and vertical 2. When the monitor is in the upright and horizontal position, landscape 1 is selected to enter a horizontal viewing screen (not shown). When the monitor is in the upright and vertical position, vertical 1 is selected to enter a vertical viewing screen 1920. When the monitor is in the upright and horizontal position, landscape 2 is selected to enter an inverted rotated 180° horizontal viewing screen 1930. When the monitor is in the upright and vertical position, vertical 2 is selected to enter an inverted 180° vertical viewing screen 1940.

FIGS. 20A-D and 33A-E illustrate display-setup displays 2000. Once “display” is selected in the menu selection list 1510, a display-setup screen 2010 is shown. The display-setup screen 2010 has a setup selection list 2005 including view, trend, contrast, language, default and vertical layout. Each item of the list 2005 also has its own selections. View allows a user to select a pleth and signal IQ view 2012, a pleth only view 2014 or a numbers view 2016. The pleth and signal IQ view 2012 shows the SpO₂ and pulse rate numbers on the left or top of the screen. The plethysmograph and signal IQ waveforms are on the right, two-thirds or bottom of the screen. The screen also indicates the signal strength of the measured signal as a perfusion index (PI). The PI is calculated as the relation of arterial pulsatile signal to the non-pulsatile signal component. The percentage measurements of methemoglobin (HbMet) and carboxyhemoglobin (HbCO) are displayed in the middle upper third of the screen above the PI measurement. The PVI measurement is displayed under the HbCO measurement. The pleth only view 2014 shows the SpO₂ and pulse rate numbers on the left or top of the screen. The plethysmograph waveform is on the right, two-thirds or bottom of the screen. The screen also indicates the signal strength of the measured signal as a perfusion index (PI). The PI is calculated as the relation of arterial pulsatile signal to the non-pulsatile signal component. The percentage measurements of methemoglobin (HbMet) and carboxyhemoglobin (HbCO) are displayed in the middle upper third of the screen above the PI measurement. The PVI measurement is displayed under the HbCO measurement. The numbers view 2016 shows the SpO₂ and pulse rate numbers and the signal IQ in the form of a pulse bar on the screen. The screen also indicates the signal strength of the measured signal as a perfusion index (PI). The PI is calculated as the relation of arterial pulsatile signal to the non-pulsatile signal component. The percentage measurements of methemoglobin (HbMet) and carboxyhemoglobin (HbCO) are displayed in the middle of the screen in line with the PI measurement. The PVI measurement is displayed under the PI measurement.

Also shown in FIGS. 20A-D, vertical layout 2020 allows a user to select a default vertical layout 2022 or a traditional vertical layout 2024. The default vertical layout 2022 displays the alarm parameters in vertical sequence of SpO₂, HbMet, HbCO, BPM, PVI and PI. The traditional vertical layout 2024 displays the alarm parameters in vertical sequence of SpO₂, BPM, PVI, PI, HbMet and HbCO. System default 2030 allows a user to select user mode or reset the settings to factory defaults having selections of low % SpO₂ limit, save last, save as adult, save as neo and restore factory. Low % SpO₂ allows the qualified user to set a custom default minimum low SpO₂ limit. When set, it will be the lowest value that the low SpO₂ alarm limit can be set to. For example, if the limit is set to 85%, then it cannot be set lower than 85% through the main Alarm menu. The unit will return to this setting after a power cycle. Save last allows the user to either use custom setting or adult/neonatal settings. “Yes” is selected to use custom settings. Save as adult stores current settings as adult default setting. Save as neo stores current settings as neonatal default setting. Restore factory recalls factory setting for custom, adult and neonatal.

Further shown in FIGS. 20A-D, Language 2040 allows a user to select the language displayed on the screen. Contrast 2050 allows a user to select contrast level. Trend 2060 allows a user to select and view the trend data between SpO₂ and BPM, PI, HbMet, HbCO or PVI, BPM and HbMet, HbCO or PVI, PI and HbMet, HbCO or PVI, HbMet and HbCO, HbMet or PVI, and HbCO and PVI. Trend 2060 also allows a user to select and view the trend data of a single SpO₂, BPM, PI, PVI, HbMet and HbCO.

FIGS. 21 and 34A-B illustrate general displays 2100. Once “general” is selected in the menu selection list 1510, a general settings display 2110 is shown. The general settings display 2110 has a selection list 2105 including average time, FastSat, home use, interface alarms, SatShare numbers, power save, smart tone and PVI. Each item of the list 2105 also has its own selections. As shown in FIG. 21, average time is selected 2110 to set the signal averaging time. In one embodiment, the time can be set to 2, 4, 8, 10, 12, 14 or 16 seconds. With FastSat the averaging time is dependent on the input signal. For the 2 and 4 second settings, the averaging times may range from 2-4 and 4-6 seconds, respectively. FastSat is selected 2120 to activate the FastSat algorithm by selecting yes. In the 2 and 4 seconds averaging mode, the FastSat algorithm is automatically enabled.

Also shown in FIG. 21, home use is selected 2130 to set the monitor to the home mode. The monitor remains in the home mode until the no setting is selected. A password is used to activate or deactivate this mode. Interface alarms is selected 2140 to enable or disable the audible alarms by selecting yes or no. The interface alarms cannot be disabled if the HbCO and HbMet parameters are present in the monitor.

Further shown in FIG. 21, SatShare numbers is selected 2150 to display the saturation and pulse rate measurements during SatShare operation by selecting a SatShare numbers setting of Yes. Power save is selected 2160 to maximize battery-operating time of the monitor while powered by the handheld battery or optional Docking Station battery. Yes is selected to disable docking station functions such as SatShare, serial and analog output. No is selected to activate these docking Station functions while operating on battery power. While operating in the power save mode, a power cycle of the monitor may be required to activate the docking station again after it has been disabled. Smart tone allows the audible pulse to continue to beep when the pleth graph shows signs of motion. Yes is selected to activate the smart tone function and no is to turn off smart tone. PVI is selected to display PVI parameter numerically on the main screen and also allows a user to change the maximum and minimum PVI settings in the trend setup menu. Yes is selected to display the PVI parameter and NO to deselect the parameter.

FIGS. 22 and 35A-B illustrate clock setup displays 2200. Once “clock” is selected in the menu selection list 1510, a clock setup display 2210 is shown. The clock display 2210 has a list 2205 including time, time format, day, month, year, day format and display clock for set-up. As shown in FIG. 22, time is selected to set the time of hour 2210, minutes 2220 and seconds 2230 in 24 hour format. Time format is selected 2240 to set the format of the time display as it will be shown on the front panel including options of 24 hour and the default of 12 hour display. Day is selected 2250 to set the numerical day. Month is selected 2260 to set the numerical month. Year is selected 2270 to set the numerical year. Day format is selected 2280 to set the format of the date display as it will be shown on the front panel including options of dd/mm/yy and the default of mm/dd/yy. Display clock is set display the date and time on the front panel by selecting yes or no.

FIGS. 23 and 36 illustrate “About” displays 2300. Once “about” is selected in the menu selection list 1510, an about display 2310 is shown. The about display 2310 displays the copyright and software versions of the handheld and docking station. The about display 2310 has a list 2305 including line frequency. A line frequency display 2320 allows a user to view the current line frequency setting without a password.

FIGS. 24 and 37 illustrate 3D alarm setup displays 2400 that enable clinicians to be alerted to changes in multiple interacting factors to provide an additional level of vigilance and flexibility to manage their patients. Once “3D Alarms” is selected in the menu selection list 1510, a 3D alarms display 2410 is shown. 3D alarm system features desat index alarm and PI delta alarm. The desat index alarm is a user-selectable feature that allows a clinician to request an audible and visual alarm if a patient experiences a specified number of desaturations over a specific period of time. The PI delta alarm is a user-selectable feature that allows a clinician to request an audible and visual alarm if perfusion at the monitored site decreases by a specified level (delta) over a specific period of time.

As shown in FIG. 24, the 3D alarms display 2410 has a list 2405 showing the desat index alarm feature including entries of desat index threshold, desat index time and desat index alarm, and the PI delta alarm feature including entries of PI delta time and PI delta alarm. In one embodiment, a desat index threshold entry 2410 has the range of 2% to 10% in 1% increment and the default is 4%. A desat index timeframe entry 2420 has the range of 1 to 4 hours in 1 hour increment and the default is 1 hour. A desat index alarm entry 2430 has the range of 1 to 25 desaturations and the default is off. A PI delta timeout entry 2440 can be set in the range of the increments of 1 min, 5 min, 30 min, 1 hour, 4 hour, 8 hour, 12 hour, 24 hour, 36 hour, 48 hour and none. The default is non. A PI delta baseline entry 2450 has selections of off, the current PI baseline or timeout.

FIGS. 25 and 38A-B illustrate configuration displays 2500. Once “config” is selected in the menu selection list 1510, a configuration password display 2510 is shown. The configuration password display 2510 allows a user to enter password by selecting soft key icons 2512 to enter a configuration display 2520. The configuration display 2520 allows a user to set line frequency to match regional power line frequency of 50 to 60 Hz that allows for cancellation of noise introduced by fluorescent lights and other sources. Default is 60 Hz.

FIGS. 26 and 39 illustrate output setup displays 2600. Once “output” is selected in the menu selection list 1510, an output display 2610 is shown. The output display 2610 has a selection list including serial, analog 1, analog 2, nurse call and polarity. As shown in FIG. 26, a serial entry 2610 has selections of serial output modes including ASCII 1, ASCII 2 and binary. In an ASCII 1 mode, ASCII text data is sent to the serial interface at one-second intervals. The ASCII text includes date and time stamp, SpO₂ pulse rate, PI, HbMet, HbCO and alarm and exception values. All text is single line followed by a line feed character and a carriage return. In an ASCII 2 mode, ASCII text data is sent to the serial interface following a query from the connecting computer. This mode will need to be active for RadNet data output. In a binary mode, compressed binary data is sent to the serial interface following a query from the connecting computer.

Also shown in FIG. 26, an analog 1 entry 2620 and analog 2 entry 2630 has selections of SpO₂ 0-100%, SpO₂ 50-100%, pulse rate, pleth, signal IQ, 0V output and 1V output. SpO₂ 0-100% scales the saturation measurement with 0% being equal to 0 volt and 100% equal to 1 volt. SpO₂ 50-100% scales the saturation measurement with 50% being equal to 0 Volt and 100% equal to 1 volt. Pulse rate scales the pulse rate measurement with 0 BPM being equal to 0 volt, and 250 BPM equal to 1 volt. Pleth traces the plethysmographic waveform as shown on the monitor display. Single IQ traces the signal IQ waveform as shown on the display. A full scale signal IQ signal (100%) is represented as 1 volt, while a zero Signal IQ signal (0%) is represented as 0 volt. 0V output is selected indicates a 0 volt calibration signal is mapped to the analog output. Use this signal for calibration of recording devices. 0 volts represents a saturation of 0% and a pulse rate of 0 bpm. 1V output is selected indicates that a 1 volt calibration signal is mapped to the analog output. Use this signal for calibration of recording devices. 1 volt represents a saturation of 100% and a pulse rate of 250 bpm.

Further shown in FIG. 26, a nurse call entry 2640 has selections of alarms, low signal IQ, and alarm and signal IQ indicating which of the event selections is based upon to activate the nurse call output. A polarity entry 2650 has selections of normal and invert. Normal polarity is a standard polarity. Invert polarity reverses the normally open and normally closed contacts.

FIGS. 27 and 40A-B illustrate service displays 2700. Once “service” is selected in the menu selection list 1510, a service password display 2710 is shown. The service password display 2710 allows a user to enter password by selecting soft key icons 2712 to enter a service display 2720. The service display 2720 has a selection list including handheld battery discharge and DS battery discharge. Handheld battery discharge is selected 2720 to deep discharge the handheld battery. DS battery discharge is selected 2730 to deep discharge the optional docking station battery. In one embodiment, the discharge cycle takes approximately 16 hours to complete for the handheld battery. The docking station battery takes approximately 30 hours to complete. A message appears in the service screen when the discharge cycle is complete. The batteries are fully charged after completion of the cycle. When deep discharge is started, the backlight automatically turns down to the default handheld battery powered level. Wait until the message changes from in progress to done.

FIGS. 28 and 41 illustrate sensitivity displays 2800 having a normal sensitivity display which is a default display 1300, a Max sensitivity display 2810 and an APOD sensitivity display 2820 showing the three sensitivity levels of normal, maximum sensitivity (Max) and adaptive probe off detection (APOD) that enable a clinician to tailor the response of a monitor to the needs of the particular patient situation. The sensitivity icon 916 in the default display 1300 (FIGS. 13A-B) is selected to toggle between the default display 1300 in a normal sensitivity mode, the Max display 2810 in a Max sensitivity mode and the APOD sensitivity display 2820 in a APOD sensitivity mode. Normal sensitivity is the recommended mode for typical monitoring purposes and is advisable for care areas where patients are observed frequently, such as ICU's. APOD is the recommended monitoring mode where there is a high probability of the sensor becoming detached. It is also the suggested mode for care areas where patients are not visually monitored continuously. This mode delivers enhanced protection against erroneous pulse rate and arterial oxygen saturation readings when a sensor becomes inadvertently detached from a patient due to excessive movement. MAX mode is recommended for patients with low perfusion or when the low perfusion message is displayed on the screen in APOD or normal sensitivity mode. This mode is not recommended for care areas where patients are not monitored visually, such as general wards. It is designed to interpret and display data at the measuring site when the signal may be weak due to decreased perfusion. When a sensor becomes detached from a patient, it will have compromised protection against erroneous pulse rate and arterial saturation readings. As shown in FIG. 28, the Max sensitivity display 2810 and the APOD sensitivity display 2820 each has a sensitivity message area 434 indicating a sensitivity mode as Max and APOD, respectively.

FIG. 29 illustrates trend toggle displays 2900 having a toggle 1 SpO₂ display 2910, a toggle 2 BPM display 2920, a toggle 3 SpMet display 2930, a toggle 4 SpCO display 2940, a toggle 5 PI display 2950 and a default display 1300. The trend toggle icon 918 in the default display 1300 (FIGS. 13A-B) is selected to toggle between the displays 2910, 2920, 2930, 2940, 2950, 1300.

A patient monitor user interface has been disclosed in detail in connection with various embodiments. These embodiments are disclosed by way of examples only and are not to limit the scope of the claims that follow. One of ordinary skill in art will appreciate many variations and modifications.

What is claimed is:

1. A patient monitor user interface comprising:
an optical sensor configured to transmit light having multiple wavelengths into a tissue site and generate a sensor signal responsive to the transmitted light after attenuation by pulsatile blood flow within the tissue site;
a patient monitor in communications with the sensor signal so as to generate multiple parameter measurements responsive to the sensor signal,
the patient monitor configured as a handheld device and including a color display, a plurality of user activated soft keys adjacent the color display and a plurality of icons provided on the color display proximate and corresponding to the soft keys;
an oxygen saturation measurement expressed as a first number;
a pulse rate expressed as a second number; and
a carboxyhemoglobin measurement expressed as a third number, wherein
the first through third numbers are presented on the color display as a first through a third color, respectively.

2. The patient monitor user interface according to claim 1 further comprising:
a methemoglobin measurement expressed as a fourth number, wherein
the fourth number is presented on the color display as fourth color.

3. The patient monitor user interface according to claim 2 wherein:
the first color is white, the second color is off-white, the third color is orange to orange-red and the fourth color is yellow to gold.

4. The patient monitor user interface according to claim 3 further comprising a pleth waveform presented on the color display as a first graph.

5. The patient monitor user interface according to claim 4 further comprising signal quality waveform presented on the color display as a second graph.

6. A patient monitor user interfacing method comprising:
providing a color display on a handheld patient monitoring device;
deriving a plurality of physiological parameter from a noninvasive multiple wavelength optical sensor attached to a patient tissue site and in communications with the handheld patient monitoring device;
presenting measurements of the physiological parameters on the color display as a selectable one of a plurality of views, wherein
a first one of the views presents the physiological parameter measurements, a plethysmograph waveform and a signal quality waveform.

7. The patient monitor user interfacing method according to claim 6 wherein a second one of the views presents the physiological parameter measurements and a pulsating height signal quality bar.

8. The patient monitor user interfacing method according to claim 7 wherein a third one of the views presents the physiological parameter measurements and a trend graph of a selected one of the physiological parameter measurements.

9. The patient monitor user interfacing method according to claim 8 wherein the physiological parameter measurements comprise carboxyhemoglobin and methemoglobin.

10. The patient monitor user interfacing method according to claim 9 wherein the carboxyhemoglobin measurement is presented in a first color and the methemoglobin measurement is presented in a second color.

11. A patient monitor user interface comprising:
an optical sensor configured to transmit light having multiple wavelengths into a tissue site and generate a sensor signal responsive to the transmitted light after attenuation by pulsatile blood flow within the tissue site;
a handheld patient monitor in communications with the sensor signal so as to generate multiple parameter measurements responsive to the sensor signal;
a color display means integrated with the handheld patient monitor for presenting the multiple parameter measurements to a user; and
a hierarchical menu means for selecting views for presenting the parameter measurements.

12. The patient monitor user interface according to claim 11 further comprising a pleth and signal quality view means for presenting the parameter measurements in addition to waveforms.

13. The patient monitor user interface according to claim 12 further comprising a numerical view means for presenting the parameter measurements without waveforms.

14. The patient monitor user interface according to claim 13 further comprising a trend view means for presenting the parameter measurements along with a trend graph for a selected one of the parameter measurements.
